SEQUENCE LISTING (49 sequences)

SEQ ID NO: 1 - protein, 265 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 112 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 3 - protein, 112 aa, synthetic construct
EIVLTQSPATLSLSPGERATLSCRASQGVSTSGYSYLHWYQQKPGQAPRLLIYLASYRESGVPARFSGSGSGTDFTLTISSLEPEDFAVYYCQHSRDLPLTFGGGTKVEIKR

SEQ ID NO: 4 - protein, 112 aa, synthetic construct
EIVLTQSPATLSLSPGERATLSCRASQGVSTSGYSYLHWYQQKPGQAPRLLIYLASYRASGVPARFSGSGSGTDFTLTISSLEPEDFAVYYCQHSRDLPLTFGGGTKVEIKR

SEQ ID NO: 5 - protein, 112 aa, synthetic construct
EIVLTQSPATLSLSPGERATLSCRASQGVSTSGYSYLAWYQQKPGQAPRLLIYLASYRASGVPARFSGSGSGTDFTLTISSLEPEDFAVYYCQHSRDLPLTFGGGTKVEIKR

SEQ ID NO: 6 - protein, 120 aa, synthetic construct [sequence omitted]

SEQ ID NO: 7 - protein, 120 aa, synthetic construct
QVQLVQSGVEVKKPGASVKVSCKASGYTFTNYYMYWVRQAPGQGLEWMGGINPSNGGTNFAQKFQGRVTLTTDSSTTTAYMELKSLQFDDTAVYYCARRDYRFDMGFDYWGQGTTVTVSS

SEQ ID NO: 8 - protein, 120 aa, synthetic construct
QVQLVQSGVEVKKPGASVKVSCKASGYTFTNYYMYWVRQAPGQGLEWMGGINPSNGGTNYAQKFQGRVTLTTDSSTTTAYMELKSLQFDDTAVYYCARRDYRFDMGFDYWGQGTTVTVSS

SEQ ID NO: 9 - protein, 120 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSS

SEQ ID NO: 10 - protein, 120 aa, synthetic construct
EVQLLESGGGLVQPGGSLRLSCAASGFTFTNYYMYWVRQAPGKGLEWVSGINPSNGGTNYADKFKGRFTISRDNSKNTLYLQMNSLRAEDTAVYYCARRDYRFDMGFDYWGQGTLVTVSS

SEQ ID NO: 11 - protein, 120 aa, synthetic construct
EVQLLESGGGLVQPGGSLRLSCAASGFTFTNYYMYWVRQAPGKGLEWVSGINPSNGGTNYADKFKGRFTLSTDSSKNTLYLQMNSLRAEDTAVYYCARRDYRFDMGFDYWGQGTLVTVSS

SEQ ID NO: 12 - protein, 120 aa, synthetic construct
EVQLLESGGGLVQPGGSLRLSCAASGFTFTNYYMYWVRQAPGKGLEWMGGINPSNGGTNYADKFKGRFTISRDNSKNTLYLQMNSLRAEDTAVYYCARRDYRFDMGFDYWGQGTLVTVSS

SEQ ID NO: 13 - protein, 120 aa, synthetic construct
EVQLLESGGGLVQPGGSLRLSCAASGFTFTNYYMYWVRQAPGKGLEWMGGINPSNGGTNYADKFKGRFTLSTDSSKNTLYLQMNSLRAEDTAVYYCARRDYRFDMGFDYWGQGTLVTVSS

SEQ ID NO: 14 - protein, 120 aa, synthetic construct
EVQLLESGGGLVQPGGSLRLSCAASGFTFTNYYMYWVRQAPGKGLEWVSGINPSNGGTNFNDSVKGRFTISRDNSKNTLYLQMNSLRAEDTAVYYCARRDYRFDMGFDYWGQGTLVTVSS

SEQ ID NO: 15 - protein, 120 aa, synthetic construct
EVQLLESGGGLVQPGGSLRLSCAASGFTFTNYYMYWVRQAPGKGLEWVSGINPSNGGTNFADKFKGRFTISRDNSKNTLYLQMNSLRAEDTAVYYCARRDYRFDMGFDYWGQGTLVTVSS

SEQ ID NO: 16 - protein, 120 aa, synthetic construct
EVQLLESGGGLVQPGGSLRLSCAASGFTFTNYYMYWVRQAPGKGLEWVSGINPSNGGTNFADKFKGRFTISRDSSKNTLYLQMNSLRAEDTAVYYCARRDYRFDMGFDYWGQGTLVTVSS

SEQ ID NO: 17 - protein, 120 aa, synthetic construct
EVQLLESGGGLVQPGGSLRLSCAASGFTFTNYYMYWVRQAPGKGLEWVSGINPSNGGTNFADKFKGRFTISTDSSKNTLYLQMNSLRAEDTAVYYCARRDYRFDMGFDYWGQGTLVTVSS

SEQ ID NO: 18 - protein, 120 aa, synthetic construct
EVQLLESGGGLVQPGGSLRLSCAASGFTFTNYYMYWVRQAPGKGLEWVSGINPSNGGTNFADKFKGRFTLSTDSSKNTLYLQMNSLRAEDTAVYYCARRDYRFDMGFDYWGQGTLVTVSS

SEQ ID NO: 19 - protein, 15 aa, synthetic construct
RASKGVSTSGYSYLH

SEQ ID NO: 20 - protein, 15 aa, synthetic construct
RASQGVSTSGYSYLH

SEQ ID NO: 21 - protein, 15 aa, synthetic construct
RASQGVSTSGYSYLA

SEQ ID NO: 22 - protein, 8 aa, synthetic construct
YLASYLES

SEQ ID NO: 23 - protein, 8 aa, synthetic construct
YLASYRES

SEQ ID NO: 24 - protein, 8 aa, synthetic construct
YLASYRAS

SEQ ID NO: 25 - protein, 9 aa, synthetic construct
QHSRDLPLT

SEQ ID NO: 26 - protein, 5 aa, synthetic construct
NYYMY

SEQ ID NO: 27 - protein, 17 aa, synthetic construct
GINPSNGGTNFNEKFKN

SEQ ID NO: 28 - protein, 17 aa, synthetic construct
GINPSNGGTNFAQKFQG

SEQ ID NO: 29 - protein, 17 aa, synthetic construct
GINPSNGGTNYAQKFQG

SEQ ID NO: 30 - protein, 17 aa, synthetic construct
GINPSNGGTNYADKFKG

SEQ ID NO: 31 - protein, 17 aa, synthetic construct
GINPSNGGTNFADKFKG

SEQ ID NO: 32 - protein, 17 aa, synthetic construct
GINPSNGGTNFNDSVKG

SEQ ID NO: 33 - protein, 11 aa, synthetic construct
RDYRFDMGFDY

SEQ ID NO: 34 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 37 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 38 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 39 - protein, 95 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWH

SEQ ID NO: 40 - protein, 122 aa, Homo sapiens
QVQLVQSGVEVKKPGASVKVSCKASGYTFTSNAISWVRQAPGQGLEWMGWISTYKGKANYAQKFQDRVTLTTDSSTTTAYMELKSLQFDDTAVYYCARWRAVVGRGGGLDVWGQGTTVTVSS

SEQ ID NO: 41 - protein, 108 aa, Homo sapiens
EIVLTQSPATLSLSPGERATLSCRASQSVSSYLAWYQQKPGQAPRLLIYDASNKATGVPARFSGSGSGTDFTLTISSLEPEDFAVYYCQQSSKWPLTFGGGTKVEIKG

SEQ ID NO: 42 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 449 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 218 aa, synthetic construct
EIVLTQSPATLSLSPGERATLSCRASQGVSTSGYSYLHWYQQKPGQAPRLLIYLASYRESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 449 aa, synthetic construct
QVQLVQSGVEVKKPGASVKVSCKASGYTFTNYYMYWVRQAPGQGLEWMGGINPSNGGTNFAQKFQGRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 449 aa, synthetic construct
EVQLLESGGGLVQPGGSLRLSCAASGFTFTNYYMYWVRQAPGKGLEWVSGINPSNGGTNYADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 449 aa, synthetic construct
EVQLLESGGGLVQPGGSLRLSCAASGFTFTNYYMYWVRQAPGKGLEWVSGINPSNGGTNFADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 449 aa, synthetic construct
EVQLLESGGGLVQPGGSLRLSCAASGFTFTNYYMYWVRQAPGKGLEWMGGINPSNGGTNYADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 449 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0347474 A1
(43) Pub. Date: Nov. 5, 2026

(54) SEQUENCE OPTIMIZATION OF A PD1 BLOCKING ANTIBODY

(71) Applicant: Cugene Inc, Waltham, MA (US)

(72) Inventors: Yue-Sheng LI, Thousand Oaks, CA (US); Lingyun RUI, Weston, MA (US); Jing XU, Waltham, MA (US)

(21) Appl. No.: 19/109,999

(22) PCT Filed: Sep. 4, 2023
(86) PCT No.: PCT/US2023/031949

§ 371 Date: Mar. 7, 2025

Related U.S. Application Data
(60) Provisional application No. 63/404,599, filed on Sep. 8, 2022.

(51) Int. Cl. C07K 16/28

(52) U.S. Cl. CPC C07K 16/2818; C07K 2317/24; C07K 2317/565; C07K 2317/567; C07K 2317/76

(57) ABSTRACT

This application provides the sequences of PD1 blocking antibody optimized based on pembrolizumab by germline sequence substitutions of the CDR residues, germline sequence substitutions of the framework somatic mutations, adoption of the most prevalent and better behaving VH3 human germline family sequence as the acceptor framework. The resulting PD1 blocking antibodies, and antigen-binding fragments thereof, have a high affinity for PD1, function to inhibit PD1 with similar or equal potency as pembrolizumab, share higher sequence similarity scores to human germline sequence, hence improved degree of humanness compared to pembrolizumab, and with predicted lower hydrophobicity than pembrolizumab.

### RELATED APPLICATIONS

This application claims benefit of U.S. Provisional Application No. 63/404,599, filed on Sep. 8, 2022, incorporated in its entirety by reference herein.

### REFERENCE TO AN ELECTRONIC SEQUENCE LISTING

The contents of the electronic sequence listing (SeqListing-CUGENE OPT-PD1 AB.xml; Size: 49 Kilobytes; Production Date: Sep. 4, 2023) is herein incorporated by reference in its entirety.

### TECHNICAL FIELD

Program death protein 1 (PD1 or CD279) is an inhibitory member of the CD28 family of receptors, primarily found on lymphoid cells, including T cells, B cells and natural killer (NK) cells, as well as myeloid cells (Quezada et al., Br. J. Cancer, 108:1560-1565, 2013). Upon binding to its ligand, PD-L1, in the presence of the T-cell receptor (TCR) signaling complex, PD1 delivers an inhibitory signal that inhibits the immune response (Okazaki et al., Proc. Natl. Acad. Sci. USA. 98:13866-13871, 2001). Prolonged immune activity can lead to continuous PD1 activation, which in turn may cause T-cell anergy or exhaustion, resulting in a reduction of TCR-mediated proliferation, and immune evasion by the cancerous cells.

Immune suppression can be reversed by disrupting the local interaction between PD1 and its ligands, PD-L1/PD-L2. Monoclonal antibodies (MAbs) that sterically block the interaction between PD1 and its ligands have demonstrated great promise as a strategy for controlling and eradicating cancer by preventing immune-cell deactivation and suppression. Among several globally marketed PD1 blocking antibodies, pembrolizumab (Keytruda®; Merck Sharp & Dohme Corp.) has been approved to treat a wide variety of cancer types and receives remarkable attention due to the high degree of efficacy.

Pembrolizumab was humanized using CDR grafting technology, which incorporated the most homologous human antibody sequences available from the Research Collaboratory for Structural Bioinformatics (RCSB) protein databank as the acceptor human frameworks (Carven G J et al., U.S. Pat. No. 8,354,509B2). The frameworks encoded by GenBank accession #AB063829 and #M29469 were used as the acceptor human frameworks for the heavy chain variable domain (VH) and light chain variable domain (VL), respectively.

Human Ab genes are formed naturally in vivo through the rearrangement of germline gene segments, generally containing unique somatic hypermutations in the frameworks that are potentially immunogenic. On the other hand, unmodified germline sequences are typical among Abs of the IgM class; hence, the body may display high tolerance to germline-encoded antibodies. Consequently, human germline frameworks are considered the preferred acceptor human frameworks for antibody humanization.

GenBank accession #AB063829, which was used as the acceptor human framework for the VH of pembrolizumab, belongs to the VH1 germline family consensus group. Nonetheless, the VH3 is the most common VH family in the human repertoire and is prevalent among all human monoclonal antibodies in clinical use and currently in clinical trials (Dudgeon K et al., PNAS 109:10879-10884, 2012). Additionally, the VH3 family is widely recognized for exhibiting the best properties in terms of solubility and thermodynamic stability (Honegger A et al., Protein Eng. Des. Sel. (2009) 22:121-134).

Moreover, the VH of pembrolizumab shares only 79.6% sequence identity with its closest human germline sequence variable region exons, IGHV1-2, thus displaying a low degree of humanness (Abhinandan K R et al., J Mol Biol (2007) 369:852-62). The low sequence similarity score of pembrolizumab's VH to its closest human germline sequence can be attributed to a combination of several factors. These include the poor level of conservation between the mouse complementary-determining regions (CDRs) and their equivalent residues in the human germline sequence, the retention of the structurally important mouse framework residues to recapitulate antigen binding, and the preservation of unique somatic mutations in the human acceptor framework AB063829.

In addition to its relatively low degree of sequence humanness, pembrolizumab was one of the most hydrophobic mAbs among the 23 U.S. Food and Drug Administration (FDA) and European Medicines Agency (EMA) approved therapeutic antibodies, as determined by hydrophobic interaction chromatography (HIC) and the apparent HIC retention factors (k). Consistent with the fact that hydrophobic proteins tend to aggregate, pembrolizumab was the only mAb with over 5% aggregate content when produced under the same conditions as other 21 approved therapeutic mAbs (Goyon et al., J. Chromatogr. B (2017) 1065-1066:35-43).

The hydrophobicity potentials of pembrolizumab's variable domains was further evaluated using SSH2.0 (http://i.uestc.edu.cn/SSH2/), a tool developed to predict hydrophobic interactions utilizing Support Vector Machine (Zhou et al., Front. Genet. 13:842127, 2022). It was found that both variable chains of pembrolizumab carry a high risk of hydrophobic interactions, with probabilities of 0.97 and 0.61 for the VH and VL, respectively. An antibody is predicted to have a high risk of hydrophobic interaction if the probability is 0.5 or higher (with 1 being the maximum possible value). The presence of hydrophobic patches on the protein surface is a strong intrinsic determinant affecting protein aggregation propensity.

The current inventors are looking to improve pembrolizumab by 1) enhancing the humanness of the variable domains by substituting mouse CDR residues with their germline counterparts, and substituting unique framework somatic mutations that carried over from the human acceptor framework with germline sequences; 2) improving the developability of the antibody by adopting the VH3 human germline family sequence, which is the most prevalent and exhibits better behavior, as the acceptor framework; and 3) reducing the antibody's hydrophobicity to mitigate antibody aggregation propensity. The resulting antibodies or antibody fragments thereof are expected to possess greater humanness and superior biophysical properties compared to pembrolizumab, while fully retaining the biological activities.

### DISCLOSURE OF THE INVENTION

In accordance with the present invention, the sequences of PD1 blocking antibody pembrolizumab (also referred to herein as the Reference Antibody) was optimized by introducing germline sequence substitutions of the CDR residues, introducing germline sequence substitutions of the framework somatic mutations, and adoption of the most prevalent and better behaving VH3 human germline family sequence as the acceptor framework. The resulting PD1 blocking antibodies, and antigen-binding fragments thereof, have a high affinity for PD1, function to inhibit PD1 with similar or equal potency as pembrolizumab, share higher sequence identity to human germline sequence, hence improved degree of humanness compared to pembrolizumab. The resulting PD1 blocking antibodies, and antigen-binding fragments thereof, are also predicted to have lower hydrophobicity than pembrolizumab and consequently lowered aggregation propensity. The resulting optimized PD-1 blocking antibody can be used to treat human diseases (e.g., cancer), infections, and other disorders either as monotherapy or in a combination therapy regimen or as part of the multispecific antibodies/multifunctional fusion proteins.

In various embodiments, human germline exon V_{H}, J_{H}, V_{K} and J_{K} sequences were chosen for CDR and framework germline substitutions. For example, human germline sequences for the V_{H} domain were chosen from V_{H} exons V_{H} 1-2 or V_{H} 3-23 (Shin et al., EMBO J. 10:3641-3645, 1991), and for the joining region (J_{H}), exon J_{H}-4 (Mattila et al., Eur. J. Immunol. 25:2578-2582, 1995). In other examples, germline V_{K} exon 3D (Cox et al., Eur. J. Immunol. 24:827-836, 1994) were chosen as acceptor sequences for V_{L} domain CDR germline sequence substitutions and J_{K} exon J_{K}-4 (Hieter et al., J. Biol. Chem. 257:1516-1522, 1982) for the joining region.

In various embodiments, the antibody or antigen-binding fragment is selected from a humanized antibody, a monoclonal antibody, a recombinant antibody, a single chain antibody, a diabody, a triabody, a tetrabody, a Fab fragment, a Fab′ fragment, a Fab₂ fragment, a F(ab)′₂ fragment, a domain antibody, an IgD antibody, an IgE antibody, an IgM antibody, an IgG1 antibody, an IgG2 antibody, an IgG3 antibody, an IgG4 antibody, or an IgG1 antibody harboring mutations in the Fc region that reduce/abolish the Fc effector function. In various embodiments, the antibody is a humanized antibody. In various embodiments, optimized antibodies, and antigen-binding fragments thereof, that have a high affinity for the human PD1 protein of SEQ ID NO: 1 are provided.

In various embodiments, the optimized PD1 blocking antibody or antigen-binding fragment thereof of the present invention comprises (a) a light chain CDR1 sequence selected from SEQ ID NOs: 19-21; (b) a light chain CDR2 sequence selected from SEQ ID NOs: 22-24; (c) a light chain CDR3 sequence set forth in SEQ ID NO: 25; (d) a heavy chain CDR1 sequence set forth in SEQ ID NO: 26; (e) a heavy chain CDR2 sequence selected from SEQ ID NOs: 27-32; and (d) a heavy chain CDR3 sequence set forth in SEQ ID NO: 33.

In various embodiments, the optimized PD1 blocking antibody or antigen-binding fragment thereof of the present invention comprises either: (a) a heavy and/or light chain variable domain(s), the variable domain(s) having a set of three light chain CDR1, CDR2, and CDR3 identical, substantially identical or substantially similar to SEQ ID NOs: 19-21, 22-24, and 25, and/or a set of three heavy chain CDR1, CDR2, and CDR3 identical, substantially identical or substantially similar to SEQ ID NOs: 26, 27-32, and 33; and (b) a set of four variable region framework regions from a human immunoglobulin (IgG).

In various embodiments, the optimized PD1 blocking antibody or antigen-binding fragment thereof of the present invention, have a high affinity for PD1, function to inhibit PD1 with similar or equal potency as the Reference Antibody, share higher sequence identity to human germline sequence, hence improved degree of humanness compared to the Reference Antibody, and with predicted lower hydrophobicity than the Reference Antibody, wherein said Reference Antibody comprises the combination of light chain variable domain and heavy chain variable domain sequences set forth in SEQ ID NOs: 2 and 6; where said Reference Antibody further comprises the combination of light chain and heavy chain sequences set forth in SEQ ID NOs: 42 and 43.

In various embodiments, the optimized PD1 blocking antibody or antigen-binding fragment thereof of the present invention comprises a light chain variable region with the sequences set forth in SEQ ID NOs: 3-5, and a heavy chain variable region with the sequences set forth in SEQ ID NOs: 7-18.

In various embodiments, the optimized PD1 blocking antibody or antigen-binding fragment thereof of the present invention comprises a light chain having a sequence identical, substantially identical or substantially similar to the sequence set forth in SEQ ID NO: 44, and a heavy chain having the sequence identical, substantially identical or substantially similar to the sequences set forth in SEQ ID NOs: 45-49.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 45.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 46.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 47.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 48.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 49.

In another aspect, the present invention relates to a pharmaceutical composition comprising an isolated antibody or antigen-binding fragment of the present invention in admixture with a pharmaceutically acceptable carrier. In various embodiments, the pharmaceutical composition comprises an isolated human antibody in admixture with a pharmaceutically acceptable carrier. In various embodiments, the pharmaceutical composition is formulated for administration via a route selected from the group consisting of subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or via infusions.

In another aspect, the present invention relates to methods for enhancing the immune response to cancerous cells in a subject, comprising administering to the subject a therapeutically effective amount (either as monotherapy or in a combination therapy regimen) of an isolated antibody or antigen-binding fragment of the present invention. In various embodiments, the present invention provides for a method of treating cancerous cells in a subject, comprising administering to said subject a therapeutically effective amount (either as monotherapy or in a combination therapy regimen) of an antibody or antigen-binding fragment thereof of the present invention. In various embodiments, the cancerous cell can be selected from any cancer. In various embodiments, the cancerous cell is selected from the group consisting of ovarian cancer, lung cancer, breast cancer, gastric cancer, prostate cancer, colon cancer, renal cell cancer, glioblastoma, and melanoma.

In various embodiments, the subject previously responded to treatment with an anti-cancer therapy, but, upon cessation of therapy, suffered relapse (hereinafter “a recurrent cancer”). In various embodiments, the subject has resistant or refractory cancer. In various embodiments, the cancerous cells are immunogenic tumors (e.g., those tumors for which vaccination using the tumor itself can lead to immunity to tumor challenge).

In another aspect, the present invention relates to combination therapies designed to treat a cancer in an subject, comprising administering to the subject a therapeutically effective amount of an isolated antibody or antigen-binding fragment of the present invention, and b) one or more additional therapies selected from the group consisting of immunotherapy, chemotherapy, small molecule kinase inhibitor targeted therapy, surgery, radiation therapy, and stem cell transplantation, wherein the combination therapy provides increased cell killing of tumor cells, i.e., a synergy exists between the isolated antibody or antigen-binding fragment and the additional therapies when co-administered.

In various embodiments, the present invention relates to methods for stimulating an immune response to pathogens, toxins, and self-antigens in a subject, comprising administering to the subject a therapeutically effective amount (either as monotherapy or in a combination therapy regimen) of an isolated antibody or antigen-binding fragment of the present invention. In various embodiments, the subject has an infectious disease that is resistant to, or ineffectively treated by, treatment using conventional vaccines.

In another aspect, an isolated immunoconjugate or fusion protein comprising an antibody or antigen-binding fragment conjugated to, linked to (or otherwise stably associated with) an effector molecule is provided. In various embodiments, the effector molecule is an immunotoxin, cytokine, chemokine, therapeutic agent, or chemotherapeutic agent.

In another aspect, the antibodies or antigen-binding fragments disclosed herein may be covalently linked to (or otherwise stably associated with) an additional functional moiety, such as a label or a moiety that confers desirable pharmacokinetic properties. In various embodiments, the label is selected from the group consisting of a fluorescent label, a radioactive label, and a label having a distinctive nuclear magnetic resonance signature.

In another aspect, the present invention provides a method for detecting in vitro or in vivo the presence of human PD1 antigen in a sample, e.g., for diagnosing a human PD1-related disease.

In another aspect, provided is an isolated nucleic acid comprising the polynucleotide sequence that encodes either the heavy chain variable domain, the light chain variable domain, or both, of an antibody or antigen-binding fragment of the invention.

In another aspect, provided is an isolated nucleic acid comprising the polynucleotide sequence that encodes either the heavy chain, the light chain, or both, of an antibody of the invention.

Also provided are vectors comprising the nucleic acid of the present invention. In one embodiment the vector is an expression vector. Also provided is an isolated cell comprising the nucleic acid of the invention. In one embodiment, the cell is a host cell comprising the expression vector of the invention. In another embodiment, the cell is a hybridoma, wherein the chromosome of the cell comprises nucleic acid of the invention. Further provided is a method of making the antibody or antigen-binding fragment of the present invention comprising culturing or incubating the cell under conditions that allow the cell to express the antigen binding protein of the invention.

### BRIEF DESCRIPTION OF THE DRAWINGS

FIG. 1 depicts a comparison of the PD1 blocking activity between the Reference Antibody (P-0734) and a pembrolizumab (PBL) biosimilar in a luciferase reporter assay. FIG. 1A and FIG. 1B depict dose-dependent increases in luminescence signal and fold induction, respectively. P-0734 and PBL biosimilar share identical variable domains and have IgG1 and IgG4 isotypes, respectively.

FIG. 2 depicts (A) ELISA binding and (B-C) PD1 blocking activity of PD1 blocking antibodies, P-1148, P-1150, P-1151, and P-1153, compared to Reference Antibody P-0734, as tested in a luciferase reporter assay. FIG. 2B and FIG. 2C depict dose-dependent increases in luminescence signal and fold induction, respectively. The variable domain sequence compositions of P-1148, P-1150, P-1151, and P-1153 are listed in Table 3, and all these antibodies have identical constant regions.

FIGS. 3A-3C depict dose-dependent increases in luminescence signal of PD1 blocking antibodies, P-1127, P-1129, P-1174, and P-1271, in a luciferase reporter assay. Reference Antibody P-0734 was included for comparison. The variable domain sequence compositions of P-1127, P-1129, P-1174, and P-1271 are listed in Table 3, and all these antibodies have identical constant regions.

FIG. 4 depicts PD1 blocking activity of PD1 blocking antibodies, P-1175 and P-1181, compared to Reference Antibody P-0734, as tested in a luciferase reporter assay. FIG. 4A and FIG. 4B depict dose-dependent increases in luminescence signal and fold induction, respectively. The variable domain sequence compositions of P-1175 and P-1181 are listed in Table 3, and these antibodies have identical constant regions.

FIG. 5 depicts PD1 blocking activity of PD1 blocking antibodies, P-1175, P-1176, P-1177, and P-1178, compared to Reference Antibody P-0734, as tested in a luciferase reporter assay. FIG. 5A and FIG. 5B depict dose-dependent increases in luminescence signal and fold induction, respectively. The variable domain sequence compositions of P-1175, P-1176, P-1177, and P-1178 are listed in Table 3, and all these antibodies have identical constant regions.

FIG. 6 depicts PD1 blocking activity of PD1 blocking antibodies, P-1198, P-1199, and P-1201, compared to Reference Antibody P-0734, as tested in a luciferase reporter assay. FIG. 6A and FIG. 6B depict dose-dependent increases in luminescence signal and fold induction, respectively. The variable domain sequence compositions of P-1198, P-1199, and P-1201 are listed in Table 3, and all these antibodies have identical constant regions.

FIG. 7 depicts PD1 blocking activity of PD1 blocking antibodies P-1194, P-1201, and P-1238, compared to Reference Antibody P-0734, as tested in a luciferase reporter assay. FIG. 7A and FIG. 7B depict dose-dependent increases in luminescence signal and fold induction, respectively. The variable domain sequence compositions of P-1194, P-1201, and P-1238 are listed in Table 3, and all these antibodies have identical constant regions.

FIG. 8 depicts binding of PD1 blocking antibodies, P-1174, P-1193, P-1198, P-1199 and P-1201 to PD1⁺HEK293 cells, compared to Reference Antibody P-0734. FIGS. 8A and 8C depict dose-dependent increases in percentage of positive cells, and FIGS. 8B and 8D depict dose dependent increases in mean fluorescence intensity (MFI). The variable domain sequence compositions of P-1193, P-1198, P-1199 and P-1201 are listed in Table 3, and all these antibodies have identical constant regions.

### MODE(S) FOR CARRYING OUT THE INVENTION

The present invention relates to antigen binding proteins such as antibodies, or antigen-binding fragments thereof that specifically bind to human PD1. These PD1 antibodies, or antigen-binding fragments thereof, have a high affinity for PD1, function to inhibit PD1 with similar or equal potency as pembrolizumab, share higher sequence identity than pembrolizumab to the closest human germline sequence, consequently improved degree of humanness, display reduced predicted lower hydrophobicity, and thus, a decreased propensity for aggregation compared to pembrolizumab, and can be used to treat human diseases (e.g., cancer), infections, and other disorders mediated by PD1. Also provided are nucleic acid molecules, and derivatives and fragments thereof, comprising a sequence of polynucleotides that encode all or a portion of a polypeptide that binds to PD1, such as a nucleic acid encoding all or part of an anti-PD1 antibody, antibody fragment, or antibody derivative. Also provided are vectors and plasmids comprising such nucleic acids, and cells or cell lines comprising such nucleic acids and/or vectors and plasmids. Also provided are methods of making, identifying, or isolating antigen binding proteins that bind to human PD1, such as anti-PD1 antibodies, methods of determining whether an antigen binding protein binds to PD1, methods of making compositions, such as pharmaceutical compositions, comprising an antigen binding protein that binds to human PD1, and methods for administering an antibody, or antigen-binding fragment thereof that binds PD1 to a subject, for example, methods for treating a condition mediated by PD1.

### DEFINITIONS

Unless otherwise defined herein, scientific and technical terms used in connection with the present invention shall have meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

Polynucleotide and polypeptide sequences are indicated using standard one- or three-letter abbreviations. Unless otherwise indicated, polypeptide sequences have their amino termini at the left and their carboxy termini at the right, and single-stranded nucleic acid sequences, and the top strand of double-stranded nucleic acid sequences, have their 5′ termini at the left and their 3′ termini at the right. A particular section of a polypeptide can be designated by amino acid residue number such as amino acids 80 to 119, or by the actual residue at that site such as Ser80 to Ser119. A particular polypeptide or polynucleotide sequence also can be described based upon how it differs from a reference sequence. Polynucleotide and polypeptide sequences of particular light and heavy chain variable domains are designated L1 (“light chain variable domain 1”) and H1 (“heavy chain variable domain 1”). Antibodies comprising a light chain and heavy chain are indicated by combining the name of the light chain and the name of the heavy chain variable domains. For example, “L4H4,” indicates, for example, an antibody comprising the light chain variable domain of L4 and the heavy chain variable domain of H4.

The term “antibody” is used herein to refer to a protein comprising one or more polypeptides substantially or partially encoded by immunoglobulin genes or fragments of immunoglobulin genes and having specificity to a tumor antigen or specificity to a molecule overexpressed in a pathological state. Light chains (LC) are classified as either kappa or lambda. Heavy chains (HC) are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition.

In a full-length antibody, each heavy chain is comprised of a heavy chain variable region (abbreviated herein as HCVR or VH) and a heavy chain constant region. The heavy chain constant region is comprised of three domains, CH1, CH2 and CH3 (and in some instances, CH4). Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The extent of the framework region and CDRs has been defined. The sequences of the framework regions of different light or heavy chains are relatively conserved within a species, such as humans. The framework region of an antibody, which is the combined framework regions of the constituent light and heavy chains, serves to position and align the CDRs in three-dimensional space. Immunoglobulin molecules can be of any class (e.g., IgG, IgE, IgM, IgD, IgA and IgY), or subclass (e.g., IgG1, IgG2, IgG 3, IgG4, IgA1 and IgA2).

The term “complementarity determining regions” or “CDRs” contain the antigen-contacting residues (“antigen contacts”). Generally, antibodies comprise six CDRs: three in the VH (CDR-H1, CDR-H2, CDR-H3), and three in the VL (CDR-L1, CDR-L2, CDR-L3). CDRs occurring at amino acid residues 24-34 (CDR-L1), 50-56 (CDR-L2), 89-97 (CDR-L3), 31-35b (CDR-H1), 50-65 (CDR-H2), and 95-102 (CDR-H3) based on Kabat numbering scheme (Kabat et al., Sequences of Proteins of Immunological Interest, 5th Ed. Public Health Service, National Institutes of Health, Bethesda, MD (1991)). Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

The term “Fc region” is used to define the C-terminal region of an immunoglobulin heavy chain. The term includes native sequence Fc regions and variant Fc regions. The Fc region of immunoglobulin generally comprises two constant domains, a CH2 domain and a CH3 domain. Numbering of amino acid residues in the Fc region or constant region is according to the EU numbering system, also called the EU index, as described in Kabat, E. A. et al, Sequences of Proteins of Immunological Interest, 5th ed., Public Health Service, National Institutes of Health, Bethesda, MD (1991), NIH Publication 91-3242. The C-terminal lysine (Lys447) of the Fc region in this invention is deleted to eliminate product charge-heterogeneity due to cleavage of C-terminal lysine during bioproduction. The Fc portion of an antibody mediates several important effector functions, e.g., cytokine induction, antibody-dependent cell-mediated cytotoxicity (ADCC), phagocytosis, complement dependent cytotoxicity (CDC) and half-life/clearance rate of antibody and antigen-antibody complexes (e.g., the neonatal FcR (FcRn) binds to the Fc region of IgG at acidic pH in the endosome and protects IgG from degradation, thereby contributing to the long serum half-life of IgG). Replacements of amino acid residues in the Fc portion to alter antibody effector function are known in the art (see, e.g., Winter et al., U.S. Pat. Nos. 5,648,260 and 5,624,821). Exemplary amino acid substitutions that ablate FcyR and C1q binding and thus abolish/significantly reduce effector function wherein said amino acid substitutions are L234A, L235A and G237A.

Antibodies exist as intact immunoglobulins or as a number of well characterized fragments. Such fragments include Fab fragments, Fab′ fragments, Fab₂, F(ab)′₂ fragments, single chain Fv proteins (“scFv”) and disulfide stabilized Fv proteins (“dsFv”), that bind to the target antigen. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. While various antibody fragments are defined in terms of the digestion of an intact antibody, one of skill will appreciate that such fragments may be synthesized de novo either chemically or by utilizing recombinant DNA methodology. Thus, as used herein, the term antibody encompasses e.g., monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)₂ fragments, antibody fragments that exhibit the desired biological activity, disulfide-linked Fvs (dsFv), intrabodies, and epitope-binding fragments or antigen binding fragments of any of the above.

A “Fab fragment”, originally resulted from papain digestion of antibodies, comprises one light chain and a portion of one heavy chain that contains the VH domain and the CH1 domain that constitutes a single antigen-binding sites. The heavy chain of a Fab molecule cannot form a disulfide bond with another heavy chain molecule.

“F(ab′)₂ fragment, originally resulted from pepsin treatment of antibodies, has two antigen-binding sites and is still capable of cross-linking antigen. A “F(ab′)₂ fragment” contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649, U.S. Pat. Nos. 4,946,778 and 5,260,203, the disclosures of which are incorporated by reference.

The terms “an antigen-binding fragment” and “antigen-binding protein” as used herein means any protein that binds a specified target antigen. “Antigen-binding fragment” includes but is not limited to antibodies and binding parts thereof, such as immunologically functional fragments. An exemplary antigen-binding fragment of an antibody is the heavy chain and/or light chain CDR(s), or the heavy and/or light chain variable region.

Diabodies are bivalent antibodies comprising two polypeptide chains, wherein each polypeptide chain comprises VH and VL regions joined by a linker that is too short to allow for pairing between two regions on the same chain, thus allowing each region to pair with a complementary region on another polypeptide chain (see, e.g., Holliger et al., Proc. Natl. Acad. Sci. USA, 90:6444-48, 1993; and Poljak et al., Structure, 2:1121-23, 1994). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Bispecific antibodies or fragments can be of several configurations. For example, bispecific antibodies may resemble single antibodies (or antibody fragments) but have two different antigen binding sites (variable regions). In various embodiments bispecific antibodies can be produced by chemical techniques (Kranz et al., Proc. Natl. Acad. Sci. USA, 78:5807, 1981; by “polydoma” techniques (see, e.g., U.S. Pat. No. 4,474,893); or by recombinant DNA techniques. In various embodiments bispecific antibodies can have binding specificities for at least two different epitopes. In various embodiments the antibodies and fragments can also be heteroantibodies. Heteroantibodies are two or more antibodies, or antibody binding fragments (e.g., Fab) linked together, each antibody or fragment having a different specificity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method.

The term “chimeric antibody” as used herein refers to an antibody which has constant region residues from one species, such as human, and variable domains (which generally engage antigen binding) from another species, such as a murine antibody that specifically binds targeted antigen.

The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “humanized antibody” as used herein refers to an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework, and such substitutions are herein referred to as back-mutations. Humanized or other monoclonal antibodies can have additional conservative amino acid substitutions which have substantially no effect on antigen binding or other immunoglobulin functions.

The term “recombinant human antibody”, as used herein, is intended to include all human antibodies that are prepared, expressed, created or isolated by recombinant means, such as antibodies expressed using a recombinant expression vector transfected into a host cell; antibodies isolated from a recombinant, combinatorial human antibody library; antibodies isolated from an animal (e.g., a mouse) that is transgenic for human immunoglobulin genes; or antibodies prepared, expressed, created or isolated by any other means that involves splicing of human immunoglobulin gene sequences to other DNA sequences. Such recombinant human antibodies have variable and constant regions derived from human germline immunoglobulin sequences. In various embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. All such recombinant means are well known to those of ordinary skill in the art.

The term “epitope” as used herein includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another. Once a desired epitope on an antigen is determined, it is possible to generate antibodies to that epitope, e.g., using the techniques described in the present disclosure. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen.

An antigen binding protein, including an antibody, “specifically binds” to an antigen if it binds to the antigen with a high binding affinity as determined by a dissociation constant (K_{D}, or corresponding Kb, as defined below) value of at least 1×10⁻⁶ M, or at least 1×10⁻⁷ M, or at least 1×10⁻⁸ M, or at least 1×10⁻⁹ M, or at least 1×10⁻¹⁰ M, or at least 1×10⁻¹¹ M. An antigen binding protein that specifically binds to the human antigen of interest may or may not be able to bind to the same antigen of interest from other species, with the same or different affinities. The term “K_{D}” as used herein refers to the equilibrium dissociation constant of a particular antibody-antigen interaction.

The term “immunogenicity” as used herein refers to the ability of an antibody or antigen binding fragment to elicit an immune response (humoral or cellular) when administered to a recipient and includes, for example, the human anti-mouse antibody (HAMA) response. A HAMA response is initiated when T-cells from a subject make an immune response to the administered antibody. The T-cells then recruit B-cells to generate specific “anti-antibody” antibodies.

The term “immune cell” as used herein means any cell of hematopoietic lineage involved in regulating an immune response against an antigen (e.g., an autoantigen). In various embodiments, an immune cell is, e.g., a T cell, a B cell, a dendritic cell, a monocyte, a natural killer cell, a macrophage, Langerhan's cells, or Kuffer cells.

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any poly-amino acid including, but not limited to, peptide mimetics such as amino acids joined by an ether as opposed to an amide bond.

The term “recombinant polypeptide”, as used herein, is intended to include all polypeptides, including fusion molecules that are prepared, expressed, created, derived from, or isolated by recombinant means, such as polypeptides expressed using a recombinant expression vector transfected into a host cell.

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
Alanine (A), Serine(S), and Threonine (T)
Aspartic acid (D) and Glutamic acid (E)
Asparagine (N) and Glutamine (Q)
Arginine (R) and Lysine (K)
Isoleucine (I), Leucine (L), Methionine (M), and Valine (V)
Phenylalanine (F), Tyrosine (Y), and Tryptophan (W)

A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within ±2 is included. In various embodiments, those that are within ±1 are included, and in various embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−. 1); glutamate (+3.0.+−. 1); serine (+0.3); asparagine (+0.2); glutamine (+0.2); glycine (0); threonine (−0.4); proline (−0.5.+−. 1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within +2 is included, in various embodiments, those that are within ±1 are included, and in various embodiments, those within ±0.5 are included. Exemplary amino acid substitutions are set forth in Table 1.

TABLE 1

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |
| | Ala, Norleucine | |

The term “polypeptide fragment” and “truncated polypeptide” as used herein refers to a polypeptide that has an amino-terminal and/or carboxy-terminal deletion as compared to a corresponding full-length protein. In various embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant” and “polypeptide mutant” as used herein refers to a polypeptide that comprises an amino acid sequence wherein one or more amino acid residues are inserted into, deleted from and/or substituted into the amino acid sequence relative to another polypeptide sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Variants of the present disclosure include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix.

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The terms “substantial similarity” or “substantially similar,” in the context of polypeptide sequences, indicate that a polypeptide region has a sequence with at least 70%, typically at least 80%, more typically at least 85%, or at least 90% or at least 95% sequence similarity to a reference sequence. For example, a polypeptide is substantially similar to a second polypeptide, for example, where the two peptides differ by one or more conservative substitution(s).

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a polypeptide or a polynucleotide) is a molecule that by virtue of its origin or source of derivation (1) is not associated with naturally associated components that accompany it in its native state, (2) is substantially free of other molecules from the same species, (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc.

The terms “label” or “labeled” as used herein refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

As used herein, the term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as PD1, PD-L1, OX-40, CD137, GITR, LAG3, TIM-3, and VISTA; treatment using bispecific T cell engaging antibodies (BITE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

The term “immunoconjugate” or “fusion protein” as used herein refers to a molecule comprising an antibody or antigen-binding fragment thereof conjugated (or linked) directly or indirectly to an effector molecule. The effector molecule can be a detectable label, an immunotoxin, cytokine, chemokine, therapeutic agent, or chemotherapeutic agent. The antibody or antigen-binding fragment thereof may be conjugated to an effector molecule via a peptide linker. An immunoconjugate and/or fusion protein retains the immunoreactivity of the antibody or antigen-binding fragment, e.g., the antibody or antigen-binding fragment has approximately the same, or only slightly reduced, ability to bind the antigen after conjugation as before conjugation. As used herein, an immunoconjugate may also be referred to as an antibody drug conjugate (ADC). Because immunoconjugates and/or fusion proteins are originally prepared from two molecules with separate functionalities, such as an antibody and an effector molecule, they are also sometimes referred to as “chimeric molecules.”

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

The terms “treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, and remission (whether partial or total). Also encompassed by “treatment” is a reduction of pathological consequence of a proliferative disease. The methods of the invention contemplate any one or more of these aspects of treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to NHL and other cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### Antibodies

In one aspect, the invention provides isolated optimized PD1 blocking antibodies, or antigen-binding fragments thereof, which specifically binds human PD-1. In certain embodiments, antibody variants having one or more amino acid substitutions are provided. Sites of interest for substitutional mutagenesis include the CDRs and FRs.

In various embodiments, human germline exon V_{H}, J_{H}, V_{K} and J_{K} sequences were chosen for CDR and framework germline substitutions. For example, human germline sequences for the V_{H} domain were chosen from V_{H} exons V_{H} 1-2 or V_{H} 3-23 (Shin et al., EMBO J. 10:3641-3645, 1991), and for the joining region (J_{H}), exon J_{H}-4 (Mattila et al., Eur. J. Immunol. 25:2578-2582, 1995). In other examples, germline V_{K} exon 3D (Cox et al., Eur. J. Immunol. 24:827-836, 1994) were chosen as acceptor sequences for V_{L} domain CDR germline sequence substitutions and J_{K} exon J_{K}-4 (Hieter et al., J. Biol. Chem. 257:1516-1522, 1982) for the joining region.

In various embodiments, the antibody or antigen-binding fragment is selected from a humanized antibody, a monoclonal antibody, a recombinant antibody, a single chain antibody, a diabody, a triabody, a tetrabody, a Fab fragment, a Fab′ fragment, a Fab₂ fragment, a F(ab)′₂ fragment, a domain antibody, an IgD antibody, an IgE antibody, an IgM antibody, an IgG1 antibody, an IgG2 antibody, an IgG3 antibody, an IgG4 antibody, or an IgG1 antibody harboring mutations in the Fc region that abolish the Fc effector function. In various embodiments, the antibody is a humanized antibody. In various embodiments, optimized antibodies, and antigen-binding fragments thereof, that have a high affinity for the human PD1 protein of SEQ ID NO: 1 are provided.

Methods for humanizing antibodies have been described in the art. The most common antibody humanization approach is to graft the 6 CDRs from a murine antibody onto a human antibody acceptor framework. The choice of human variable regions, both light and heavy, to be used in making the desired humanized antibodies is very important to reduce immunogenicity. U.S. Pat. No. 5,693,761 to Queen et al. refined the antibody humanization approaches by selecting the human variable region with the highest sequence identity to provide the framework sequences for the humanizing project. Human Ab genes are formed in vivo by rearrangement of germline gene segments and contains unique somatic mutations; consequently, unmodified germline-encoded antibody sequences (e.g., Tomlinson, I. M., et al., J. Mol. Biol. 227:776-798, 1992; and Cox, J. P. L. et al., Eur. J. Immunol. 24:827-836, 1994; the contents of each of which are expressly incorporated herein by reference) are considered preferred acceptor human frameworks.

CDR grafting often results in partial or complete loss of affinity of the humanized antibody, and some residues from the murine framework sequences need to be retained to replace the human residues at the corresponding positions in order to restore some of the lost affinity. Candidate residues for retention are typically those adjacent in linear sequence to a CDR or physically within 6 Å of any CDR residue. Alternatively, U.S. Pat. No. 5,821,337 to Carter et al, and U.S. Pat. No. 5,859,205 to Adair et al disclose specific Kabat residue positions in the framework, which, in a humanized antibody may require substitution with the correspondent mouse amino acid to preserve antibody activity. The number of residues from the framework of the parental antibodies should be minimized by determining the importance of particular framework amino acid residues experimentally (e.g., assaying antigen-binding) in the humanized antibodies to ensure the highest degree of humanness.

The current inventors adopted similar approaches but with some key distinctions; not only the framework residues, but also some CDR residues will be substituted with their respective counterparts from the closest human germline, in order to improve the humanness of the antibody and thus reduce its immunogenicity potential. Early findings evinced a view that the conformation of the CDRs was exquisitely sensitive to the chemical environment of the surrounding framework. Thus, avoidance of any perturbation of the CDRs has been an overriding principle in the design of humanized Abs. However, transfer of the entire CDRs with poor level of conservation between the mouse CDRs and their equivalent residues in the closest human germline sequence increase the mouse sequence content in the resulting humanized antibody. Since only a limited number of amino acid positions within the CDRs are directly involved in antigen binding, it is deemed feasible to replace some CDR residues with their counterpart residues from the closest human germline sequence without compromising antibody activity, regardless of whether such substitutions are conservative or non-conservative. The choice of CDR residues to be substituted with their equivalent residues in human germline hinges considerably on the probability of an amino acid at a particular position interacting with the antigen. Therefore, utilizing a three-dimensional immunoglobulin structure or model in complex with the antigen permits analysis of the paratope. Such analysis helps exclude from modification any CDR residues that are directly engaged in antigen binding, or any adjacent conserved residues.

The DNA sequence encoding the variable region of the antibody is then fused to human constant region sequences. The sequences of human constant regions may be found in Kabat et al. (1991) Sequences of Proteins of Immunological Interest, N.I.H. publication no. 91-3242. The choice of isotype will be guided by the desired effector functions, such as complement fixation, or activity in antibody-dependent cellular cytotoxicity. In various embodiments, the isotype is selected from the group consisting of IgG1, IgG2, IgG3 and IgG4. Either of the human light chain constant regions, kappa or lambda, may be used.

Antibodies or antigen-binding fragments thereof of the invention can comprise any constant region known in the art. The light chain constant region can be, e.g., a human kappa- or lambda-type light chain constant region. The heavy chain constant region can be, for example, an alpha-, delta-, epsilon-, gamma-, or mu-type heavy chain constant regions, e.g., an IgA-, IgD-, IgE-, IgG- and IgM-type heavy chain constant region. In various embodiments, the light or heavy chain constant region is a fragment, derivative, variant, or mutein of a naturally occurring constant region.

In certain alternative embodiments, the antibodies of the present invention can be engineered by modifying one or more residues in one or both variable regions (i.e., V_{H} and/or V_{L}) to improve binding affinity and/or decreases immunogenicity, or by modifying residues within the constant region(s) for purposes of altering the serum half-life, complement fixation, Fc receptor binding, and/or antigen-dependent cellular cytotoxicity of the antibody. In various embodiments, the antibodies may be modified for purposes of modifying the glycosylation of the antibody. Methods for performing each of the modifications described herein, and others, are well known to the skilled artisan.

In various embodiments, the invention provides an isolated optimized PD1 blocking antibody, or antigen-binding fragment thereof, which specifically binds human PD1 and comprises: (a) a light chain CDR1 sequence identical, substantially identical or substantially similar to a CDR1 sequence selected from SEQ ID NOs: 19-21; (b) a light chain CDR2 sequence identical, substantially identical or substantially similar to a CDR2 sequence selected from SEQ ID NOs: 22-24; (c) a light chain CDR3 sequence identical, substantially identical or substantially similar to a CDR3 sequence selected from SEQ ID NO: 25; (d) a heavy chain CDR1 sequence identical, substantially identical or substantially similar to a CDR1 sequence selected from SEQ ID NO: 26; (e) a heavy chain CDR2 sequence identical, substantially identical or substantially similar to a CDR2 sequence selected from SEQ ID NOs: 27-32; (f) a heavy chain CDR3 sequence identical, substantially identical or substantially similar to a CDR3 sequence selected from SEQ ID NO: 33.

In various embodiments, the invention provides an isolated optimized PD1 blocking antibody, or antigen-binding fragment thereof, which specifically binds human PD1 and comprises: (a) the light chain CDR1, CDR2, and CDR3 sequences set forth in SEQ ID NO: 20, SEQ ID NO: 23, and SEQ ID NO: 25 and the heavy chain CDR1, CDR2, and CDR3 sequences set forth in SEQ ID NO: 26, SEQ ID NO: 28, and SEQ ID NO: 33; or (b) the light chain CDR1, CDR2, and CDR3 sequences set forth in SEQ ID NO: 20, SEQ ID NO: 23, and SEQ ID NO: 25 and the heavy chain CDR1, CDR2, and CDR3 sequences set forth in SEQ ID NO: 26, SEQ ID NO: 30, and SEQ ID NO: 33; or (c) the light chain CDR1, CDR2, and CDR3 sequences set forth in SEQ ID NO: 20, SEQ ID NO: 23, and SEQ ID NO: 25 and the heavy chain CDR1, CDR2, and CDR3 sequences set forth in SEQ ID NO: 26, SEQ ID NO: 31, and SEQ ID NO: 33.

In various embodiments, the invention provides an isolated optimized PD1 blocking antibody, or antigen-binding fragment thereof, which specifically binds human PD1 and comprises: (a) a light chain variable region comprising amino acids having the sequence set forth in SEQ ID NO: 3, and a heavy chain variable region comprising amino acids having the sequence set forth in SEQ ID NO: 7; or (b) a light chain variable region comprising amino acids having the sequence set forth in SEQ ID NO: 3, and a heavy chain variable region comprising amino acids having the sequence set forth in SEQ ID NO: 9; or (c) a light chain variable region comprising amino acids having the sequence set forth in SEQ ID NO: 3, and a heavy chain variable region comprising amino acids having the sequence set forth in SEQ ID NO: 11; (d) a light chain variable region comprising amino acids having the sequence set forth in SEQ ID NO: 3, and a heavy chain variable region comprising amino acids having the sequence set forth in SEQ ID NO: 13; or (e) a light chain variable region comprising amino acids having the sequence set forth in SEQ ID NO: 3, and a heavy chain variable region comprising amino acids having the sequence set forth in SEQ ID NO: 18.

In various embodiments, the optimized PD1 blocking antibody or antigen-binding fragment thereof of the present invention comprises a light chain having a sequence identical, substantially identical or substantially similar to the sequence set forth in SEQ ID NO: 44, and a heavy chain having the sequence identical, substantially identical or substantially similar to the sequences set forth in SEQ ID NOs: 45-49.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 45.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 46.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 47.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 48.

In various embodiments, an isolated humanized antibody or antigen-binding fragment thereof of the present invention binds to human PD1 and comprises the light chain sequence set forth in SEQ ID NO: 44, and the heavy chain sequence set forth in SEQ ID NO: 49.

### Pharmaceutical Compositions

In one aspect, the present invention provides a pharmaceutical composition comprising an antibody or antigen-binding fragment thereof as described above. The pharmaceutical compositions, methods and uses of the invention thus also encompass embodiments of combinations (co-administration) with other active agents, as detailed below.

Generally, the antibodies, or antigen-binding fragments thereof of the present invention are suitable to be administered as a formulation in association with one or more pharmaceutically acceptable excipient(s). The term ‘excipient’ is used herein to describe any ingredient other than the compound(s) of the invention. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody. Pharmaceutical compositions of the present invention and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP conditions.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Any method for administering peptides, proteins or antibodies accepted in the art may suitably be employed for the antibodies and portions of the invention.

The pharmaceutical compositions of the invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions, and kidney dialytic infusion techniques. Various embodiments include the intravenous and the subcutaneous routes.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed, sustained, pulsed, controlled, targeted and programmed release.

For example, in one aspect, sterile injectable solutions can be prepared by incorporating the anti-PD1 antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies of the invention can also be administered intranasally or by inhalation, typically in the form of a dry powder (either alone, as a mixture, or as a mixed component particle, for example, mixed with a suitable pharmaceutically acceptable excipient) from a dry powder inhaler, as an aerosol spray from a pressurized container, pump, spray, atomizer (preferably an atomizer using electrohydrodynamics to produce a fine mist), or nebulizer, with or without the use of a suitable propellant, or as nasal drops.

The pressurized container, pump, spray, atomizer, or nebulizer generally contains a solution or suspension of an antibody of the invention comprising, for example, a suitable agent for dispersing, solubilizing, or extending release of the active, a propellant(s) as solvent.

Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the compound of the invention, a suitable powder base and a performance modifier.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release. Modified release formulations include delayed, sustained, pulsed, controlled, targeted and programmed release.

In the case of dry powder inhalers and aerosols, the dosage unit is determined by means of a valve which delivers a metered amount. Units in accordance with the invention are typically arranged to administer a metered dose or “puff” of an antibody of the invention. The overall daily dose will typically be administered in a single dose or, more usually, as divided doses throughout the day.

The antibodies and antibody portions of the invention may also be formulated for an oral route administration. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Pharmaceutical compositions intended for oral use may be prepared according to any method known to the art for the manufacture of pharmaceutical compositions and such compositions may contain one or more agents selected from the group consisting of sweetening agents in order to provide a pharmaceutically elegant and palatable preparation. For example, to prepare orally deliverable tablets, the antibody or antigen-binding fragment thereof is mixed with at least one pharmaceutical excipient, and the solid formulation is compressed to form a tablet according to known methods, for delivery to the gastrointestinal tract. The tablet composition is typically formulated with additives, e.g., a saccharide or cellulose carrier, a binder such as starch paste or methyl cellulose, a filler, a disintegrator, or other additives typically used in the manufacture of medical preparations. To prepare orally deliverable capsules, DHEA is mixed with at least one pharmaceutical excipient, and the solid formulation is placed in a capsular container suitable for delivery to the gastrointestinal tract. Compositions comprising antibodies or antigen-binding fragments thereof may be prepared as described generally in Remington's Pharmaceutical Sciences, 18th Ed. 1990 (Mack Publishing Co. Easton Pa. 18042) at Chapter 89, which is herein incorporated by reference.

In various embodiments, the pharmaceutical compositions are formulated as orally deliverable tablets containing antibodies or antigen-binding fragments thereof in admixture with non-toxic pharmaceutically acceptable excipients which are suitable for manufacture of tablets. These excipients may be inert diluents, such as calcium carbonate, sodium carbonate, lactose, calcium phosphate or sodium phosphate, granulating and disintegrating agents, for example, maize starch, gelatin or acacia, and lubricating agents, for example, magnesium stearate, stearic acid, or talc. The tablets may be uncoated, or they may be coated with known techniques to delay disintegration and absorption in the gastrointestinal track and thereby provide a sustained action over a longer period of time. For example, a time delay material such as glyceryl monostearate or glyceryl distearate alone or with a wax may be employed.

In various embodiments, the pharmaceutical compositions are formulated as hard gelatin capsules wherein the antibody or antigen-binding fragment thereof is mixed with an inert solid diluent, for example, calcium carbonate, calcium phosphate, or kaolin or as soft gelatin capsules wherein the antibody or antigen-binding fragment thereof is mixed with an aqueous or an oil medium, for example, arachis oil, peanut oil, liquid paraffin or olive oil.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

### Therapeutic And Diagnostic Uses

In another aspect, the present invention relates to methods for enhancing the immune response to cancerous cells in a subject, comprising administering to the subject a therapeutically effective amount (either as monotherapy or in a combination therapy regimen) of an isolated antibody or antigen-binding fragment of the present invention. In various embodiments, the present invention provides for a method of treating cancerous cells in a subject, comprising administering to said subject a therapeutically effective amount (either as monotherapy or in a combination therapy regimen) of an antibody or antigen-binding fragment thereof of the present invention. In various embodiments, the cancerous cell is associated with elevated expression of PD1.

Cancerous cells that can be treated according to the invention include sarcomas and carcinomas. In various embodiments, the cancerous cell is selected from the group of tumors including but not limited to ovarian cancer, lung cancer, breast cancer, gastric cancer, prostate cancer, colon cancer, renal cell cancer, glioblastoma, and melanoma.

In various embodiments, the subject previously responded to treatment with an anti-cancer therapy, but, upon cessation of therapy, suffered relapse (hereinafter “a recurrent cancer”). In various embodiments, the subject has resistant or refractory cancer. In various embodiments, the cancerous cells are immunogenic tumors (e.g., those tumors for which vaccination using the tumor itself can lead to immunity to tumor challenge).

In various embodiments, the present antibodies and antigen-binding fragments thereof can be utilized to directly kill or ablate cancerous cells in vivo. Direct killing involves administering the antibodies (which are optionally fused to a cytotoxic drug) to a subject requiring such treatment. In various embodiments, the cancer comprises cancer cells expressing PD1 at a higher level than noncancerous cells of a comparable tissue. Since the antibodies recognize PD1 on cancer cells, any such cells to which the antibodies bind are destroyed. Where the antibodies are used alone to kill or ablate cancer cells, such killing or ablation can be affected by initiating endogenous host immune functions, such as CDC and/or ADCC. Assays for determining whether an antibody kills cells in this manner are within the purview of those skilled in the art.

In various embodiments, the present antibodies and antigen-binding fragments thereof can be utilized to promote growth inhibition and/or proliferation of a cancerous tumor cell. These methods may inhibit or prevent the growth of the cancer cells of said subject, such as for example, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. As a result, where the cancer is a solid tumor, the modulation may reduce the size of the solid tumor by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

The inhibition of the cancer cell proliferation can be measured by cell-based assays, such as bromodeoxyuridine (BRDU) incorporation (Hoshino et al., Int. J. Cancer 38, 369, 1986; Campana et al., J. Immunol. Meth. 107:79, 1988; [³H]-thymidine incorporation (Chen, J., Oncogene 13:1395-403, 1996; Jeoung, J., J. Biol. Chem. 270:18367-73, 1995; the dye Alamar Blue (available from Biosource International) (Voytik-Harbin et al., In Vitro Cell Dev Biol Anim 34:239-46, 1998). The anchorage independent growth of cancer cells is assessed by colony formation assay in soft agar, such as by counting the number of cancer cell colonies formed on top of the soft agar (see Examples and Sambrook et al., Molecular Cloning, Cold Spring Harbor, 1989).

The inhibition of cancer cell growth in a subject may be assessed by monitoring the cancer growth in a subject, for example in an animal model or in human subjects. One exemplary monitoring method is tumorigenicity assays. In one example, a xenograft comprises human cells from a pre-existing tumor or from a tumor cell line. Tumor xenograft assays are known in the art and described herein (see, e.g., Ogawa et al., Oncogene 19:6043-6052, 2000). In another embodiment, tumorigenicity is monitored using the hollow fiber assay, which is described in U.S. Pat. No. 5,698,413, which is incorporated herein by reference in its entirety.

The percentage of the inhibition is calculated by comparing the cancer cell proliferation, anchorage independent growth, or cancer cell growth under modulator treatment with that under negative control condition (typically without modulator treatment). For example, where the number of cancer cells or cancer cell colonies (colony formation assay), or PRDU or [³H]-thymidine incorporation is A (under the treatment of modulators) and C (under negative control condition), the percentage of inhibition would be (C−A)/C×100%.

“Therapeutically effective amount” or “therapeutically effective dose” refers to that amount of the therapeutic agent being administered which will relieve to some extent one or more of the symptoms of the disorder being treated.

A therapeutically effective dose can be estimated initially from cell culture assays by determining an IC₅₀. A dose can then be formulated in animal models to achieve a circulating plasma concentration range that includes the IC₅₀ as determined in cell culture. Such information can be used to more accurately determine useful doses in humans. Levels in plasma may be measured, for example, by HPLC. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

For administration to human subjects, the total monthly dose of the antibodies or antigen-binding fragments thereof of the disclosure can be in the range of 0.5-1200 mg per subject, 0.5-1100 mg per subject, 0.5-1000 mg per subject, 0.5-900 mg per subject, 0.5-800 mg per subject, 0.5-700 mg per subject, 0.5-600 mg per subject, 0.5-500 mg per subject, 0.5-400 mg per subject, 0.5-300 mg per subject, 0.5-200 mg per subject, 0.5-100 mg per subject, 0.5-50 mg per subject, 1-1200 mg per subject, 1-1100 mg per subject, 1-1000 mg per subject, 1-900 mg per subject, 1-800 mg per subject, 1-700 mg per subject, 1-600 mg per subject, 1-500 mg per subject, 1-400 mg per subject, 1-300 mg per subject, 1-200 mg per subject, 1-100 mg per subject, or 1-50 mg per subject depending, of course, on the mode of administration. For example, an intravenous monthly dose can require about 1-1000 mg/subject. In various embodiments, the antibodies or antigen-binding fragments thereof of the disclosure can be administered at about 1-200 mg per subject, 1-150 mg per subject or 1-100 mg/subject. The total monthly dose can be administered in single or divided doses and can, at the physician's discretion, fall outside of the typical ranges given herein.

An exemplary, non-limiting daily dosing range for a therapeutically or prophylactically effective amount of an antibody or antigen-binding fragment thereof of the disclosure can be 0.001 to 100 mg/kg, 0.001 to 90 mg/kg, 0.001 to 80 mg/kg, 0.001 to 70 mg/kg, 0.001 to 60 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, or 1 to 1 mg/kg body weight. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In various embodiments, the total dose administered will achieve a plasma antibody concentration in the range of, e.g., about 1 to 1000 μg/ml, about 1 to 750 μg/ml, about 1 to 500 μg/ml, about 1 to 250 μg/ml, about 10 to 1000 μg/ml, about 10 to 750 μg/ml, about 10 to 500 μg/ml, about 10 to 250 μg/ml, about 20 to 1000 μg/ml, about 20 to 750 μg/ml, about 20 to 500 μg/ml, about 20 to 250 μg/ml, about 30 to 1000 μg/ml, about 30 to 750 μg/ml, about 30 to 500 μg/ml, about 30 to 250 μg/ml.

Toxicity and therapeutic index of the pharmaceutical compositions of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

In various embodiments, single or multiple administrations of the pharmaceutical compositions are administered depending on the dosage and frequency as required and tolerated by the subject. In any event, the composition should provide a sufficient quantity of at least one of the antibodies or antigen-binding fragments thereof disclosed herein to effectively treat the subject. The dosage can be administered once but may be applied periodically until either a therapeutic result is achieved or until side effects warrant discontinuation of therapy.

The dosing frequency of the administration of the antibody or antigen-binding fragment thereof pharmaceutical composition depends on the nature of the therapy and the particular disease being treated. The subject can be treated at regular intervals, such as weekly or monthly, until a desired therapeutic result is achieved. Exemplary dosing frequencies include, but are not limited to once weekly without break; once weekly, every other week; once every 2 weeks; once every 3 weeks; weakly without break for 2 weeks, then monthly; weakly without break for 3 weeks, then monthly; monthly; once every other month; once every three months; once every four months; once every five months; or once every six months, or yearly.

### Combination Therapy

As used herein, the terms “co-administration”, “co-administered” and “in combination with”, referring to the antibodies or antigen-binding fragments thereof of the disclosure and one or more other therapeutic agents, is intended to mean, and does refer to and include the following: simultaneous administration of such combination of antibodies or antigen-binding fragments thereof of the disclosure and therapeutic agent(s) to a subject in need of treatment, when such components are formulated together into a single dosage form which releases said components at substantially the same time to said subject; substantially simultaneous administration of such combination of antibodies or antigen-binding fragments thereof of the disclosure and therapeutic agent(s) to a subject in need of treatment, when such components are formulated apart from each other into separate dosage forms which are taken at substantially the same time by said subject, whereupon said components are released at substantially the same time to said subject; sequential administration of such combination of antibodies or antigen-binding fragments thereof of the disclosure and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of antibodies or antigen-binding fragments thereof of the disclosure and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

In another aspect, the present invention relates to combination therapies designed to treat a cancer in an subject, comprising administering to the subject a therapeutically effective amount of an isolated antibody or antigen-binding fragment of the present invention, and b) one or more additional therapies selected from the group consisting of immunotherapy, chemotherapy, small molecule kinase inhibitor targeted therapy, surgery, radiation therapy, and stem cell transplantation, wherein the combination therapy provides increased cell killing of tumor cells, i.e., a synergy exists between the isolated antibody or antigen-binding fragment and the additional therapies when co-administered.

In various embodiments, the immunotherapy is selected from the group consisting of: treatment using agonistic, antagonistic, or blocking antibodies to co-stimulatory or co-inhibitory molecules (immune checkpoints) such as CTLA-4, PD-L1, OX-40, CD137, GITR, LAG3, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF and IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

A wide array of conventional compounds have been shown to have anti-neoplastic activities. These compounds have been used as pharmaceutical agents in chemotherapy to shrink solid tumors, prevent metastases and further growth, or decrease the number of malignant T-cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When the antibody or antigen-binding fragment disclosed herein is administered in combination with another conventional anti-neoplastic agent, either concomitantly or sequentially, such antibody or antigen-binding fragment may enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant T-cells.

Pharmaceutical compounds that may be used for combinatory anti-tumor therapy comprises a chemotherapeutic agent selected from the group consisting of: daunorubicin, dactinomycin, doxorubicin, bleomycin, mitomycin, nitrogen mustard, chlorambucil, melphalan, cyclophosphamide, 6-mercaptopurine, 6-thioguanine, bendamustine, cytarabine (CA), 5-fluorouracil (5-FU), floxuridine (5-FUdR), methotrexate (MTX), colchicine, vincristine, vinblastine, etoposide, teniposide, cisplatin, carboplatin, oxaliplatin, pentostatin, cladribine, cytarabine, gemcitabine, pralatrexate, mitoxantrone, diethylstilbestrol (DES), fluradabine, ifosfamide, hydroxyureataxanes (such as paclitaxel and doxetaxel) and/or anthracycline antibiotics, as well as combinations of agents such as, but not limited to, DA-EPOCH, CHOP, CVP or FOLFOX.

In various embodiments, the small molecule kinase inhibitor targeted therapy comprises a small molecule kinase inhibitor selected from the group consisting of Bruton's tyrosine kinase (BTK) inhibitor, phosphatidylinositol-3-kinase (PI3K) inhibitor, SYK inhibitor (e.g., entospletinib), AKT inhibitor, mTOR inhibitor, Src inhibitor, JAK/STAT inhibitor, Ras/Raf/MEK/ERK inhibitor, and Aurora inhibitor (see, D'Cruz et al, Expert Opin Pharmacother, 14(6): 707-21, 2013).

In various embodiments, the combination therapy comprises administering the antibody or antigen-binding fragment thereof and the one or more additional therapies simultaneously. In various embodiments, the antibody or antigen-binding fragment thereof composition and the one or more additional therapies are administered sequentially, i.e., the antibody or antigen-binding fragment thereof composition is administered either prior to or after the administration of the one or more additional therapies.

In various embodiments, the administrations of the antibody or antigen-binding fragment thereof composition and the one or more additional therapies are concurrent, i.e., the administration period of the antibody or antigen-binding fragment thereof composition and the one or more additional therapies overlap with each other.

In various embodiments, the administrations of the antibody or antigen-binding fragment thereof composition and the one or more additional therapies are non-concurrent. For example, in various embodiments, the administration of the antibody or antigen-binding fragment thereof composition is terminated before the one or more additional therapies is administered. In various embodiments, the administration of the one or more additional therapies is terminated before the antibody or antigen-binding fragment thereof composition is administered.

When the antibody or antigen-binding fragment thereof disclosed herein is administered in combination with one or more additional therapies, either concomitantly or sequentially, such antibody or antigen-binding fragment thereof may enhance the therapeutic effect of the one or more additional therapies or overcome cellular resistance to the one or more additional therapies. This allows for decreased dosage or duration of the one or more additional therapies, thereby reducing the undesirable side effects, or restores the effectiveness of the one or more additional therapies.

### Immunoconjugates

The application further provides immunoconjugates comprising an antibody or antigen-binding fragment thereof of the present invention conjugated (or linked) directly or indirectly to an effector molecule. In this regard, the term “conjugated” or “linked” refers to making two polypeptides into one contiguous polypeptide molecule. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. In various embodiments, an antibody or antigen-binding fragment is joined to an effector molecule. In other embodiments, an antibody or antigen-binding fragment joined to an effector molecule is further joined to a lipid, a protein or peptide to increase its half-life in the body. Accordingly in various embodiments, the antibodies of the present disclosure may be used to deliver a variety of effector molecules.

The effector molecule can be a detectable label, an immunotoxin, cytokine, chemokine, therapeutic agent, or chemotherapeutic agent.

Specific, non-limiting examples of immunotoxins include, but are not limited to, abrin, ricin, Pseudomonas exotoxin (PE, such as PE35, PE37, PE38, and PE40), diphtheria toxin (DT), botulinum toxin, cholix toxin, or modified toxins thereof, or other toxic agents that directly or indirectly inhibit cell growth or kill cells.

A “cytokine” is class of proteins or peptides released by one cell population which act on another cell as intercellular mediators. Cytokines can act as an immune-modulating agent. Examples of cytokines include lymphokines, monokines, growth factors and traditional polypeptide hormones. Thus, embodiments may utilize an interferon (e.g., IFN-α, IFN-β, and IFN-γ); tumor necrosis factor super family (TNFSF) member; human growth hormone; thyroxine; insulin; proinsulin; relaxin; prorelaxin; follicle stimulating hormone (FSH); thyroid stimulating hormone (TSH); luteinizing hormone (LH); hepatic growth factor; prostaglandin, fibroblast growth factor; prolactin; placental lactogen, OB protein; TNF-α; TNF-β; integrin; thrombopoietin (TPO); a nerve growth factor such as NGF-β; platelet-growth factor; TGF-α; TGF-β; insulin-like growth factor-I and -II; erythropoietin (EPO); colony stimulating factors (CSFs) such as macrophage-CSF (M-CSF); granulocyte-macrophage-CSF (GM-CSF); and granulocyte-CSF (G-CSF); an interleukin (e.g., IL-2, IL-7, IL-15, IL-18, and IL-21), kit-ligand or FLT-3, angiostatin, thrombospondin, or endostatin. These cytokines include native and modified cytokines.

Chemokines can also be conjugated to the antibodies disclosed herein. Chemokines are a superfamily of small (approximately about 4 to about 14 kDa), inducible and secreted pro-inflammatory cytokines that act primarily as chemoattractants and activators of specific leukocyte cell subtypes. Chemokine production is induced by inflammatory cytokines, growth factors and pathogenic stimuli. The chemokine proteins are divided into subfamilies (alpha, beta, and delta) based on conserved amino acid sequence motifs and are classified into four highly conserved groups—CXC, CC, C and CX3C, based on the position of the first two cysteines that are adjacent to the amino terminus. To date, more than 50 chemokines have been discovered and there are at least 18 human seven-transmembrane-domain (7TM) chemokine receptors. Chemokines of use include, but are not limited to, RANTES, MCAF, MCP-1, and fractalkine.

The therapeutic agent can be a chemotherapeutic agent. Useful chemotherapeutic agents for the preparation of immunoconjugates include auristatin, dolastatin, MMAE, MMAF, AFP, DM1, AEB, doxorubicin, daunorubicin, methotrexate, melphalan, chlorambucil, vinca alkaloids, 5-fluorouridine, mitomycin-C, taxol, L-asparaginase, mercaptopurine, thioguanine, hydroxyurea, cytarabine, cyclophosphamide, ifosfamide, nitrosoureas, cisplatin, carboplatin, mitomycin, dacarbazine, procarbazine, topotecan, nitrogen mustards, cytoxan, etoposide, BCNU, irinotecan, camptothecins, bleomycin, idarubicin, dactinomycin, plicamycin, mitoxantrone, asparaginase, vinblastine, vincristine, vinorelbine, paclitaxel, and docetaxel and salts, solvents and derivatives thereof.

The effector molecules can be linked to an antibody or antigen-binding fragment of the present invention using any number of means known to those of skill in the art. Both covalent and noncovalent attachment means may be used. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids. In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

Procedures for conjugating the antibodies with the effector molecules have been previously described and are within the purview of one skilled in the art.

An immunoconjugate of the present invention retains the immunoreactivity of the antibody or antigen-binding fragment, e.g., the antibody or antigen-binding fragment has approximately the same, or only slightly reduced, ability to bind the antigen after conjugation as before conjugation. As used herein, an immunoconjugate is also referred to as an antibody drug conjugate (ADC).

### Diagnostic Uses

In another aspect, the present invention provides a method for detecting in vitro or in vivo the presence of human PD1 antigen in a sample, e.g., for diagnosing a human PD1-related disease. In some methods, this is achieved by contacting a sample to be tested, along with a control sample, with a human sequence antibody or a human monoclonal antibody of the invention, or an antigen-binding portion thereof (or a bispecific or multispecific molecule), under conditions that allow for formation of a complex between the antibody and human PD1. Complex formation is then detected (e.g., using an ELISA) in both samples, and any statistically significant difference in the formation of complexes between the samples is indicative the presence of human PD1 antigen in the test sample.

In various embodiments, methods are provided for detecting cancer or confirming the diagnosis of cancer in a subject. In various embodiments, the antibodies or antigen-binding fragments can be labeled or unlabeled for diagnostic purposes. In various embodiments, the antibody or antigen-binding fragment provided herein may also be used in a method of detecting the susceptibility of a mammal to certain diseases. In various embodiments, the antibodies or antigen-binding fragments are attached to a label that is able to be detected (e.g., the label can be a radioisotope, fluorescent compound, enzyme or enzyme co-factor).

### Multispecific Antibody

In certain embodiments, an antibody provided herein is a multispecific antibody, e.g., a bispecific antibody. Multispecific antibodies are monoclonal antibodies that have binding specificities for at least two different sites. In certain embodiments, one of the binding specificities is for PD1 and the other is for any other antigen. In certain embodiments, bispecific antibodies may bind to two different epitopes of PD1. Bispecific antibodies may also be used to localize cytotoxic agents to cells which express PD1. Bispecific antibodies can be prepared as full-length antibodies or antibody fragments. Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al, EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M. et al Science 229 (1985) 81-83); using leucine zippers to produce bi-specific antibodies (see, e.g., Kostelny, S. A. et al, J. Immunol. 148 (1992) 1547-1553; using “diabody” technology for making bispecific antibody fragments (see, e.g., Holliger, P. et al, Proc. Natl. Acad. Sci. USA 90 (1993) 6444-6448); and using single-chain Fv (scFv) dimers (see, e.g., Gruber, M et al, J. Immunol. 152 (1994) 5368-5374); and preparing trispecific antibodies as described, e.g., in Tutt, A. et al, J. Immunol. 147 (1991) 60-69). Engineered antibodies with three or more functional antigen binding sites, including “Octopus antibodies,” are also included herein (see, e.g., US 2006/0025576).

### Polynucleotides and Antibody Expression

The application further provides polynucleotides comprising a nucleotide sequence encoding an anti-PD1 antibody or antigen-binding fragment thereof.

The present invention is also directed to host cells that express a PD1 polypeptide and/or the anti-PD1 antibodies of the invention. A wide variety of host expression systems known in the art can be used to express an antibody of the present invention including prokaryotic (bacterial) and eukaryotic expression systems (such as yeast, baculovirus, plant, mammalian and other animal cells, transgenic animals, and hybridoma cells), as well as phage display expression systems.

An antibody of the invention can be prepared by recombinant expression of immunoglobulin light and heavy chain genes in a host cell. To express an antibody recombinantly, a host cell is transformed, transduced, infected or the like with one or more recombinant expression vectors carrying DNA fragments encoding the immunoglobulin light and/or heavy chains of the antibody such that the light and/or heavy chains are expressed in the host cell. The heavy chain and the light chain may be expressed independently from different promoters to which they are operably-linked in one vector or, alternatively, the heavy chain and the light chain may be expressed independently from different promoters to which they are operably-linked in two vectors one expressing the heavy chain and one expressing the light chain. Optionally, the heavy chain and light chain may be expressed in different host cells.

Additionally, the recombinant expression vector can encode a signal peptide that facilitates secretion of the antibody light and/or heavy chain from a host cell. The antibody light and/or heavy chain gene can be cloned into the vector such that the signal peptide is operably-linked in-frame to the amino terminus of the antibody chain gene. The signal peptide can be an immunoglobulin signal peptide or a heterologous signal peptide. Preferably, the recombinant antibodies are secreted into the medium in which the host cells are cultured, from which the antibodies can be recovered or purified.

An isolated DNA encoding a heavy chain variable region (HCVR) can be converted to a full-length heavy chain gene by operably-linking the HCVR-encoding DNA to another DNA molecule encoding heavy chain constant regions. An isolated DNA encoding a light chain variable region (LCVR) may be converted to a full-length light chain gene (as well as to a Fab light chain gene) by operably linking the LCVR-encoding DNA to another DNA molecule encoding a light chain constant region.

In addition to the antibody heavy and/or light chain gene(s), a recombinant expression vector of the invention carries regulatory sequences that control the expression of the antibody chain gene(s) in a host cell. The term “regulatory sequence” is intended to include promoters, enhancers and other expression control elements (e.g., polyadenylation signals), as needed, that control the transcription or translation of the antibody chain gene(s). The design of the expression vector, including the selection of regulatory sequences may depend on such factors as the choice of the host cell to be transformed, the level of expression of protein desired. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and/or polyoma virus.

Additionally, the recombinant expression vectors of the invention may carry additional sequences, such as sequences that regulate replication of the vector in host cells (e.g., origins of replication) and one or more selectable marker genes. The selectable marker gene facilitates selection of host cells into which the vector has been introduced. Preferred selectable marker genes include the dihydrofolate reductase (dhfr) gene (for use in dhfr-minus host cells with methotrexate selection/amplification), the neo gene (for G418 selection), and glutamine synthetase (GS) in a GS-negative cell line (such as NSO) for selection/amplification.

For expression of the light and/or heavy chains, the expression vector(s) encoding the heavy and/or light chains is introduced into a host cell by standard techniques e.g., electroporation, calcium phosphate precipitation, DEAE-dextran transfection, transduction, infection and the like. Although it is theoretically possible to express the antibodies of the invention in either prokaryotic or eukaryotic host cells, eukaryotic cells are preferred, and most preferably mammalian host cells, because such cells are more likely to assemble and secrete a properly folded and immunologically active antibody. Preferred mammalian host cells for expressing the recombinant antibodies of the invention include Chinese Hamster Ovary (CHO cells), NSO myeloma cells, human embryonic kidney (HEK) 293 cells, COS cells, and SP2/0 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown under appropriate conditions known in the art. Antibodies can be recovered from the host cell and/or the culture medium using standard purification methods.

The invention provides a host cell comprising a nucleic acid molecule of the present invention. Preferably a host cell of the invention comprises one or more vectors or constructs comprising a nucleic acid molecule of the present invention. For example, a host cell of the invention is a cell into which a vector of the invention has been introduced, said vector comprising a polynucleotide encoding a LCVR of an antibody of the invention and/or a polynucleotide encoding a HCVR of the invention. The invention also provides a host cell into which two vectors of the invention have been introduced; one comprising a polynucleotide encoding a LCVR of an antibody of the invention and one comprising a polynucleotide encoding a HCVR present in an antibody of the invention and each operably-linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes.

Once expressed, the intact antibodies, individual light and heavy chains, or other immunoglobulin forms of the present invention can be purified according to standard procedures of the art. Additionally, standard techniques for removing viruses from recombinantly expressed antibody preparations are also known in the art. The purification process for antibodies of the present invention may include a step of filtering to remove viruses from the mainstream of one or more chromatography operations. Substantially pure immunoglobulins of at least about 90%, about 92%, about 94% or about 96% homogeneity are preferred, and about 98 to about 99% or more homogeneity most preferred, for pharmaceutical uses. Once purified, partially or to homogeneity as desired, the sterile antibodies may then be used therapeutically, as directed herein.

In view of the aforementioned discussion, the present invention is further directed to an antibody obtainable by a process comprising the steps of culturing a host cell including, but not limited to a mammalian, plant, bacterial, transgenic animal, or transgenic plant cell which has been transformed by a polynucleotide or a vector comprising nucleic acid molecules encoding antibodies of the invention so that the nucleic acid is expressed and, optionally, recovering the antibody from the host cell culture medium.

Host cells can also be used to produce portions, or fragments, of intact antibodies, e.g., Fab fragments or scFv molecules by techniques that are conventional. For example, it may be desirable to transfect a host cell with DNA encoding either the light chain or the heavy chain of an antibody of this invention. Recombinant DNA technology may also be used to remove some or all the DNA encoding either or both of the light and heavy chains that is not necessary for binding to human PD1. The molecules expressed from such truncated DNA molecules are also encompassed by the antibodies of the invention.

Methods for expression of single chain antibodies and/or refolding to an appropriate active form, including single chain antibodies, from bacteria such as E. coli have been described and are well-known and are applicable to the antibodies disclosed herein (see, e.g., Buchner et al., Anal. Biochem. 205:263-270, 1992; Pluckthun, Biotechnology 9:545, 1991; Huse et al., Science 246:1275, 1989 and Ward et al., Nature 341:544, 1989, all incorporated by reference herein).

The following examples are offered to more fully illustrate the invention, but are not construed as limiting the scope thereof.

### Example 1

### PD1 Blocking Antibody Sequence Optimization

The current invention aims to optimize the variable domain sequences of pembrolizumab to enhance the score of similarity to the human germline sequences, a measure for their “humanness”. This enhancement could potentially lower the immunogenicity risk. Additionally, the inventors used a human V_{H}3 family germline sequence, which, despite being less homologous, is more prevalent and behaves better, as an alternative acceptor framework. This was done with the aim of improving the biophysical properties of the resulting humanized antibody, ensuring it retains full activity, and enhancing its sequence humanness.

Pembrolizumab was humanized by CDR grafting technology, using the most homologous human antibody sequences available in RCSB protein databank as the acceptor human frameworks. The frameworks found in GenBank under accession numbers AB063829 (SEQ ID NO: 40) and M29469 (SEQ ID NO: 41) were used as the acceptor human frameworks for the heavy chain variable domain (VH) and light chain variable domain (VL), respectively (Carven G J et al., U.S. Pat. No. 8,354,509B2). However, pembrolizumab only shares 79.6% sequence identity to IGHV1-2, its closest human germline, according to a comparison of the variable region exons using the International Immunogenetics Information System (IMGT) DomainGapAlign tool (www.imgt.org). A similarity score to the human germline sequence was proposed as a defining criterion for therapeutic antibodies by the international nonproprietary names (INN) group of WHO in 2014, presumably based on the notion that higher similarity could suggest reduced immunogenicity. The low score of similarity, or “degree of humanness” (Abhinandan K R et al., J Mol Biol (2007) 369:852-62) of pembrolizumab's heavy chain could be due to the poor level of conservation between the mouse CDRs and their human sequence equivalents, the necessity to retain a few structurally important mouse framework residues to recapitulate antigen binding, and the preservation of unique somatic mutations in the human framework sequence AB063829.

To enhance the degree of humanness of pembrolizumab, certain CDR residues were targeted for substitution with their equivalent residues from the closest human germline sequences. This method is herein referred to as CDR germlining. While avoidance of CDR perturbation has traditionally been a central principle in humanized Abs design, only a limited number of CDR residues engage in direct antigen interaction. Therefore, certain CDR residues can be replaced without compromising the activity of the antibody. According to the Kabat numbering scheme, CDRs are defined as amino acid residues 24-34 (CDR-L1), 50-56 (CDR-L2), 89-97 (CDR-L3), 31-35b (CDR-H1), 50-65 (CDR-H2), and 95-102 (CDR-H3).

As for the CDR3 sequences, a portion of the light chain CDR3 (CDR-L3) and the entire heavy chain CDR3 (CDR-H3) were not part of the germline sequence's variable region exons V region. Consequently, there are no human germline residues available to replace the mouse CDR counterparts. Additionally, CDR3s, especially CDR-H3, are highly variable and vital for antigen binding and functional activity, making it crucial to preserve their conformations. As such, both CDR-L3 (QHSRDLPLT; SEQ ID NO: 25) and CDR-H3 (RDYRFDMGFDY; SEQ ID NO: 33) of pembrolizumab were excluded from the CDR germlining process.

The sequences of pembrolizumab's CDR-L1 and CDR-L2 were aligned to their counterparts from the closest human germline IGKV3D-11 (GenBank accession #X17264; SEQ ID NO: 39). The alignments are shown in Table 2A. Likewise, the alignments of the CDR-H1 and CDR-H2 sequences of pembrolizumab with the closest human germline sequence, IGHV1-2 (GenBank accession #X62106; SEQ ID NO: 37), are displayed in Table 2B.

TABLE 2A
Alignments of the CDR-L1 and CDR-L2 sequences
of pembrolizumab with IGKV3D-11

| Antibody | CDR-L1 | CDR-L2 |
|---|---|---|
| Pembrolizumab | RASKGVSTSGYSYLH | LASYLES |
| IGKV3D-11 | RASQGV----SSYLA | DASNRAT |

TABLE 2B
Alignments of the CDR-H1 and CDR-H2
sequences of pembrolizumab with IGHV1-2

| Antibody | CDR-H1 | CDR-H2 |
|---|---|---|
| Pembrolizumab | NYYMY | GINPSNGGTNFNEKFKN |
| IGHV1-2 | GYYMH | WINPNSGGTNYAQKFQG |

“-” indicates sequence gap.
Residues in bold and italic represent those interacting with PD1, as per the complex structure (Horita S. et al., Sci Rep (2016) 6:35297).
Underscored residues are subject to CDR germlining process.

Multiple pembrolizumab CDR residues directly engage polar interactions with PD1, such as hydrogen bond and salt bridge (Horita, S. et al. Sci. Rep. (2016) 6:35297). The contacting residues in or around both VL and VH CDR1 and CDR2 include ^{L}Ser28, ^{L}Tyr30 in CDR-L1, ^{L}Tyr49 immediately prior to CDR-L2, ^{L}Tyr53 in CDR-L2, ^{H}Tyr33, ^{H}Tyr35 in CDR-H1, ^{H}Asn52, ^{H}Ser53, ^{H}Asn54, ^{H}Thr57, ^{H}Asn58 in CDR-H2 (where the superscripted letter ‘L’ donates light chain, and ‘H’ refers to heavy chain). Except for ^{L}Tyr49, which is not a CDR residue and not shown, all the antigen-interacting CDR residues mentioned above are in bold and italic in Tables 2A and 2B. Among the CDR residues that differ from the germline sequences, CDR-L1 residues ^{L}Lys27, ^{L}His34, CDR-L2 residues ^{L}Leu54, ^{L}Glu55, CDR-H2 residues ^{H}Phe59, ^{H}Asn60, ^{H}Glu61, ^{L}Lys64, and ^{H}Asn65 (underscored in Tables 2A and 2B) were selected for CDR germlining. They were replaced with their respective human germline equivalents with the following amino acid substitutions: ^{L}K27Q, ^{L}H34A, ^{L}L54R, ^{L}E55A, ^{H}F59Y, ^{H}N60A, ^{H}E61Q, ^{H}K64Q, and ^{H}N65G either individually or in combination. Other CDR residues were reserved to avoid any disruption of the antigen-interacting residues.

For CDR-H1 (NYYMY; SEQ ID NO: 26), only a single residue, ^{H}Asn31, is eligible for CDR germlining, to be replaced with its equivalent residue, glycine, in the human germline sequence. Other residues were kept either because they engage in direct antigen interaction with PD1 (^{H}Tyr33 and ^{H}Tyr35) or because they are conserved between the mouse and human germline sequences (^{H}Tyr32 and ^{H}Met34). However, considering CDR-H1's short length of only five amino acids and the fact that two residues directly interact with the antigen, any amino acid alteration might disrupt the CDR confirmation and affect the antibody's activity. Consequently, ^{H}Asn31 was not modified through CDR germlining, and CDR-H1 is fully reserved.

In addition to the low level of conservation between the mouse CDRs and their corresponding human germline sequences, the pembrolizumab heavy chain framework (FRs) also contain multiple non-germline residues. They arose due to the retention of the unique somatic mutations in the acceptor framework sequence AB063829. These somatic mutations, including ^{H}Val9 in FR-H1, ^{H}Thr76, ^{H}Lys82a, ^{H}Gln83, ^{H}Phe84 in FR-H3 and ^{H}Thr108 in FR-H4, are not considered structurally significant. Replacement with their respective germline counterparts, ^{H}V9A, ^{H}T76S, ^{H}K82aS, ^{H}Q83R, ^{H}F84S, ^{H}T108L, will lead to a considerable improvement in the similarity score to the human germline sequence, without disrupting the CDR conformation or altering the antibody's activity.

Moreover, IGHV3-23 (SEQ ID NO: 38) was used as an alternative acceptor framework to investigate whether utilizing a human acceptor framework with substantially lower sequence homology but superior biophysical attributes could enhance the biophysical properties of the resultant humanized antibody without compromising its functional activity. IGHV3-23 belongs to the human antibody heavy chain germline VH3 family, which is the most common VH family in the human repertoire. It is the most prevalent among the marketed human monoclonal antibodies and is widely acknowledged for its superior drug-like properties. Given that the CDR conformation was exquisitely sensitive to the chemical environment of the surrounding framework, a few structurally significant framework residues in pembrolizumab, which differ from their VH3 germline family equivalents, were targeted for reverse mutation to their corresponding pembrolizumab equivalents. Furthermore, several CDR-H2 residues were selected for CDR germlining using IGHV3-23 CDR-H2 as a template to improve the similarity score to the human germline sequence.

Alignments of the CDR-H1 and CDR-H2 sequence of pembrolizumab with IGHV3-23 are displayed in Table 2C. Six CDR-H2 residues, ^{H}Phe59, ^{H}Asn60, ^{H}Glu61, ^{H}Lys62, ^{H}Phe63, and ^{H}Asn65 (underscored in Table 2C; the superscripted letter ‘L’ denotes light chain, and ‘H’ refers to heavy chain), were selected for CDR germlining with the following amino acid substitutions: ^{H}F59Y, ^{H}N60A, ^{H}E61D, ^{H}K62S, ^{H}F63V, and ^{H}N65G. CDR-H1 was excluded from CDR germlining for the reason described earlier.

TABLE 2C
Alignments of the CDR-H1 and CDR-H2
sequences of pembrolizumab with IGHV3-23

| Antibody | CDR-H1 | CDR-H2 |
|---|---|---|
| Pembrolizumab | NYYMY | GINPSNGGTNFNEKFKN |
| IGHV3-23 | SYAMS | AISGSGGSTYYADSVKG |

Residues in bold and italic represent those interacting with PD1, as per the complex structure (Horita S. et al., Sci Rep (2016) 6:35297).
Underscored residues are selected for CDR germlining process

Two framework residues, ^{H}Thr30 and ^{H}Arg94, were considered structurally significant and were preserved without alteration to their corresponding germline equivalents, ^{H}Ser30 and ^{H}Lys94. Five additional IGHV3 framework residues, ^{H}Val48, ^{H}Ser49, ^{H}Ile69, ^{H}Arg71, and ^{H}Asn73, which belong to the vernier zone (U.S. Pat. Nos. 5,821,337 and 5,859,205) and may be of structural significance, were reverted to their corresponding pembrolizumab equivalent residues, ^{H}Met48, ^{H}Gly49, ^{H}Leu69, ^{H}Thr71, and ^{H}Ser73, either individually or in combination. The importance of particular framework amino acid residues was assessed experimentally. The number of reverse mutations was minimized to ensure the highest similarity score to the germline sequence without negatively affecting antibody activity.

All the optimized antibody sequences were expressed as full length antibody with a kappa light chain constant region containing the sequence set forth in SEQ ID NO: 34 and a modified IgG1 heavy chain constant region containing the sequence set forth in SEQ ID NO: 35. Table 3 lists the SEQ ID NOS of the VL, VH, CDR-L1, CDR-L2, and CDR-H2 of the exemplary optimized PD1 blocking antibodies along with the Reference Antibody P-0734, comprising VL and VH sequences set forth in SEQ ID NO: 2 and SEQ ID NO: 6, respectively. All antibodies of the present invention contain identical CDR-L3 (SEQ ID NO: 25), CDR-H1 (SEQ ID NO: 26), and CDR-H3 (SEQ ID NO: 33).

TABLE 3
Exemplary PD1 blocking antibodies
resulted from CDR and FR germlining

| | SEQ ID NO | | | | |
|---|---|---|---|---|---|
| PD1 Antibody | VL | VH | CDR-L1 | CDR-L2 | CDR-H2 |
| P-0734 | 2 | 6 | 19 | 22 | 27 |
| P-1127 | 3 | 6 | 20 | 23 | 27 |
| P-1129 | 4 | 6 | 20 | 24 | 27 |
| P-1148 | 4 | 7 | 20 | 24 | 28 |
| P-1150 | 5 | 7 | 21 | 24 | 28 |
| P-1151 | 4 | 8 | 20 | 24 | 29 |
| P-1153 | 5 | 8 | 21 | 24 | 29 |
| P-1174 | 3 | 7 | 20 | 23 | 28 |
| P-1175 | 2 | 10 | 19 | 22 | 31 |
| P-1176 | 2 | 11 | 19 | 22 | 31 |
| P-1177 | 2 | 12 | 19 | 22 | 31 |
| P-1178 | 2 | 13 | 19 | 22 | 31 |
| P-1181 | 2 | 14 | 19 | 22 | 30 |
| P-1193 | 3 | 15 | 20 | 23 | 32 |
| P-1194 | 3 | 11 | 20 | 23 | 31 |
| P-1198 | 3 | 16 | 20 | 23 | 32 |
| P-1199 | 3 | 17 | 20 | 23 | 32 |
| P-1201 | 3 | 18 | 20 | 23 | 32 |
| P-1238 | 3 | 13 | 20 | 23 | 32 |
| P-1271 | 3 | 9 | 20 | 23 | 28 |

### Example 2

### Construction, Production, and Purification of the Optimized PD1 Blocking Antibodies

All genes were codon-optimized for expression in mammalian cells, and they were synthesized and subsequently subcloned into their recipient mammalian expression vector by GenScript. Protein expression is driven by a CMV promoter, and a synthetic SV40 polyA signal sequence is positioned at the 3′ end of the coding sequence. A leader sequence was engineered at the N-terminus of the constructs to ensure appropriate signaling and processing for secretion.

The antibodies were produced by co-transfecting vectors harboring light chain and heavy chain with a 1:1 ratio in ExpiCHO cells (ThermoFisher), following the manufacturer's instructions. On the day of transfection, ExpiCHO cells were diluted to 6×10⁶ cells/mL in ExpiCHO™ expression medium (ThermoFisher). Expression vectors, totaling 0.8 μg DNA/mL culture volume, were mixed with cold OptiPRO™ medium (40 μL/mL cell culture). Following the addition of ExpiFectamine™ CHO reagent at 3.2 μL/mL cell culture, the solution was gently mixed and subsequently incubated for 5 min at room temperature. The ExpiFectamine™ CHO/plasmid DNA complexes were then slowly transferred to the cells and incubated at 37 °C in a shaking incubator at 130 rpm with 8% CO₂. ExpiFectamine™ CHO enhancer (6 μL/mL cell culture) and ExpiCHO™ feed (240 μL/mL cell culture) were added to the flask with gentle swirling 18-22 hours post transfection. After 8 days of cultivation, the supernatant was harvested for purification by centrifugation for 20 min at 2200 rpm, followed by sterile filtration using a 0.22 μm filter (Corning).

The secreted antibody was purified from cell culture supernatants using Protein A affinity chromatography. Cell culture supernatant was loaded onto a MabSelect SuRe 5-mL column (Cytiva) equilibrated with 5 column volumes (CV) of phosphate buffered saline, pH 7.2 (ThermoFisher). Unbound protein was removed by washing with 5 CVs of PBS, pH 7.2, and target protein was eluted with 25 mM sodium citrate, 25 mM sodium chloride buffer, pH 3.2. Antibody solution was neutralized by adding 3% of 1 M Tris buffer, pH 10.2 followed by concentration and buffer exchange to PBS, pH 7.2 using Amicon® Ultra-15 Ultracel with 10 kDa MWCO (Merck Millipore).

The purity and molecular weight of the purified antibodies were analyzed by SDS-PAGE, both with and without a reducing agent, and then stained with Coomassie (Imperial™ protein stain, ThermoFisher). The SurePAGE™ Pre-Cast gel system (8-16% Bis-Tris, GenScript) was used according to the manufacturer's instruction. The content aggregates in the antibodies was examined on an Agilent 1200 high-performance liquid chromatography (HPLC) system. Samples were injected into an AdvanceBio size-exclusion column (300Å, 4.6×150 mm, 2.7 μm, LC column, Agilent), using 150 mM sodium phosphate buffer, pH 7.0, as the mobile phase at 25 °C.

The antibody concentration of purified protein samples was determined by measuring the absorbance at 280 nm using a Nanodrop spectrophotometer (ThermoFisher) divided by the molar extinction coefficient calculated based on its amino acid sequence. Endotoxin levels of purified protein samples were measured using Endosafe nexgen-PTS (Charles River) as per the manufacturer's instruction.

### Example 3

### Assays to Evaluate the Biological Activities of the Optimized PD1 Blocking Antibodies

Antibodies of the invention were tested for their antigen binding activity by well-known methods such as enzyme-linked immunosorbent assay (ELISA). Briefly, Nunc Maxisorp plates (ThermoFisher) were coated with recombinant human PD1-6His protein in bicarbonate buffer, pH 9.4 (ThermoFisher), overnight at 4 °C, using 1 μg of antigen per well (100 μL/well). After triple washing with PBS/0.05% Tween20, plates were incubated with SuperBlock (ThermoFisher) for 2 hours at room temperature to block nonspecific binding. The PD1 antibodies, serially diluted three-fold with blocking buffer (PBS with 1% bovine serum albumin), were added to the plates (100 μL/well) post washing and incubated at room temperature for one hour. After another washing, antibodies were detected by incubating with a horseradish peroxidase (HRP)-conjugated goat anti-human IgG Fc antibody (ThermoFisher) diluted 1:5000 in blocking buffer (100 μL/well) for an hour at room temperature. After final washing, TMB substrate (ThermoFisher) at 100 μL/well was added. Plates were sealed and left to incubate in dark for 5-20 min. The reaction was stopped by adding 2N sulfuric acid (Ricca Chemical) (50 μL/well), and the absorbance was measured at 450 nm with a plate reader. The curves were plotted and the half-maximal effective concentration (EC₅₀) values were calculated using GraphPad Prism software.

Additionally, HEK 293T cells stably expressing human PD1 gene (Crown Bioscience) was used to determine the cell-based binding strength of the optimized PD1 antibodies by flow cytometry. After harvesting, HEK293-hPD1 cells were seeded onto a 96-well U-bottom plate at 1×10⁵ cells/well (100 μL), incubated with Fc block (1:50) for 20 minutes at 4 °C, and subsequently washed with FACS buffer (PBS, 1% FBS). Cells were then treated with three-fold serial dilutions of each antibody at concentrations ranging from 0.01-100 nM in FACS buffer for 30 min at 4 °C. The cells were washed twice with FACS buffer to remove unbound molecules, and 40 μL of the 1:100 diluted PE-labeled goat anti-human Fc secondary antibody (eBiosciences) was added to the cells. After 30 min incubation at 4 °C, the cells were washed twice with FACS buffer. Antibodies bound to the cells were detected with PE-labeled secondary antibody by flow cytometry (BD ACCURI-C6), and EC₅₀ values were calculated using GraphPad Prism software.

Furthermore, a thaw-and-use format of Promega luciferase reporter assay, a biologically relevant mechanistic-based assay, was used to measure the potency in blocking PD1 interaction by PD1 antibodies. Cell thawing and plating procedures were followed exactly as described in the manufacturer's protocol.

Briefly, one vial (0.5 mL) of PD-L1 aAPC/CHO-K1 cells were thawed and mixed with 14.5 mL cell recovery medium (90% Ham's F12/10% FBS). Next, 100 μL of this cell suspension was added to the inner 60 wells of two 96-well flat-bottom assay plates, while perimeter wells received 100 μL of cell recovery medium. After overnight incubation at 37 °C and 5% CO2, the medium was discarded. The inner wells received 40 μL of 3-fold serially diluted compounds, while the perimeter wells got 80 μL of assay buffer (99% RPMI 1640/1% FBS). Subsequently, one vial (0.5 mL) of PD1 effector cells were thawed and mixed with 5.9 mL of assay buffer, and 40 μL of this mixture was added to the inner wells. After a 6-hour incubation at 37 °C, 5% CO2, and a 7-minute equilibration at room temperature, 80 μL of Bio-Glo™ reagent was added to all wells. The plates were then incubated at room temperature for 10 minutes with shaking. The resulting luminescence was measured using a luminescence plate reader (BioTek synergy h1).

Background was calculated by averaging relative light units (RLU) of perimeter wells. Fold Induction was determined as the RLU of the antibody sample minus background, divided by the RLU of the control samples (without antibody) minus background, or fold induction=RLU (antibody-background))/(RLU (no antibody ctrl-background). Finally, EC50 values were determined using the curves fitted using GraphPad Prism software.

### Example 4

### Evaluation of the PD1 Antibodies Comprising Germlining Modifications Based on the Closest Human Germline Sequences

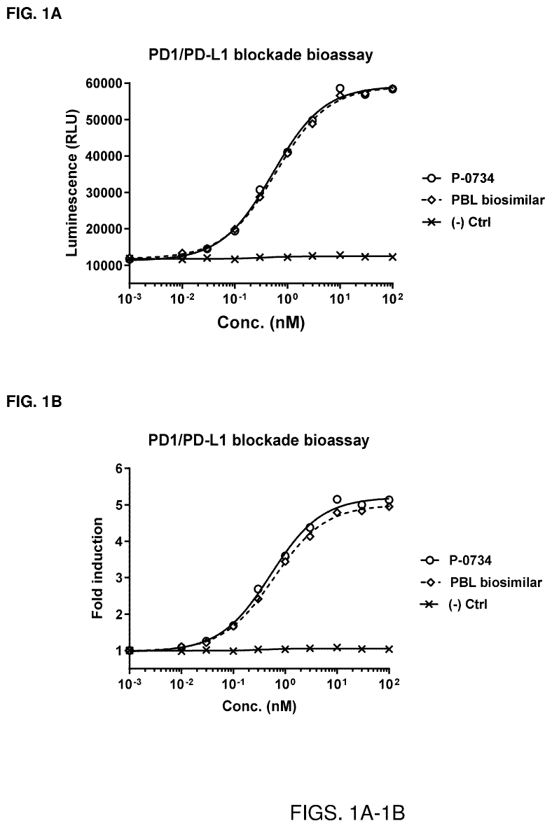

Firstly, the effectiveness of P-0734 in inhibiting the PD1/PD-L1 interaction was compared with that of pembrolizumab (PBL) biosimilar. While P-0734 and PBL biosimilar share the identical variable domains, they differ in their heavy chain constant region. The PBL contains an IgG4 constant chain containing the S228P mutation (SEQ ID NO: 36), whereas P-0734 has an IgG1 constant chain with L234A/L235A/G237A mutations (SEQ ID NO: 35) to abrogate Fc effector function. As shown in FIG. 1, P-0734 and PBL biosimilar were equally potent in blocking the interaction between PD1 and PD-L1. This result was expected and confirms that the ability to block PD1 blockade is determined by the variable domain sequences, and not by the immunoglobulin classes. P-0734 faithfully recapitulates the potency of the PBL biosimilar in blocking the PD1/PD-L1 interaction and is herein referred to as the Reference Antibody.

The impacts of CDR germlining substitutions-H34A in CDR-L1 and HF60Y in CDR-H2 were evaluated using antibodies with slightly different mutational contexts. These antibodies, P-1148, P-1150, P-1151, and P-1153, all contain germlining substitutions ^{L}K27Q in CDR-L1, ^{L}L54R, ^{L}E55A in CDR-L2, and ^{H}N60A, ^{H}E61Q, ^{H}K64Q, ^{H}N65G in CDR-H2. P-1150 includes an additional ^{L}H34A substitution in CDR-L1, P-1151 has an extra ^{H}F59Y substitution in CDR-H2, and P-1153 harbors both ^{L}H34A and ^{H}F59Y changes in addition. Table 4 provides a list of the CDR germlining substitutions for these exemplary PD1 blocking antibodies.

TABLE 4
CDR germlining substitutions of exemplary PD1 blocking antibodies

| PD1 Ab | CDR-L1 | CDR-L2 | CHR-H2 |
|---|---|---|---|
| P-1148 | ^{L}K27Q | ^{L}L54R, ^{L}E55A | ^{H}N60A, ^{H}E61Q, ^{H}K64Q, ^{H}N65G |
| P-1150 | ^{L}K27Q, ^{L}H34A | ^{L}L54R, ^{L}E55A | ^{H}N60A, ^{H}E61Q, ^{H}K64Q, ^{H}N65G |
| P-1151 | ^{L}K27Q | ^{L}L54R, ^{L}E55A | ^{H}F59Y, ^{H}N60A, ^{H}E61Q, ^{H}K64Q, ^{H}N65G |
| P-1153 | ^{L}K27Q, ^{L}H34A | ^{L}L54R, ^{L}E55A | ^{H}F59Y, ^{H}N60A, ^{H}E61Q, ^{H}K64Q, ^{H}N65G |

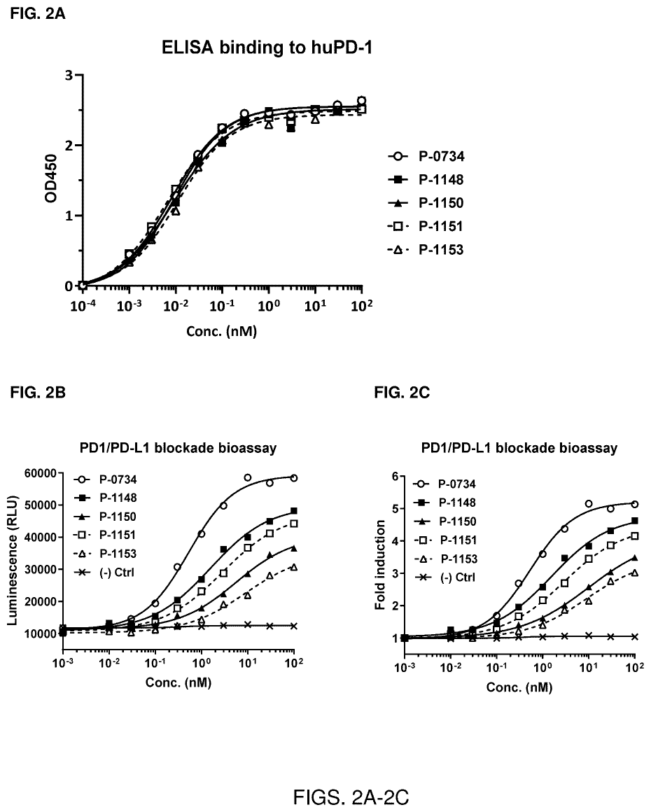

As illustrated in FIGS. 2B & 2C and summarized in Table 5, the CDR-L1 germlining substitution ^{L}H34A consistently led to a reduction in PD1 blockade potency (EC₅₀) of approximately 3.5-fold, along with a 25% reduction in both Vmax and fold induction, regardless of the presence (P-1151 vs P-1153) or absence (P-1148 vs P-1150) of the ^{H}F59Y substitution. The impact of ^{H}F59Y germlining substitution was similarly assessed, with data shown in FIGS. 2B & 2C and summarized in Table 5. Regardless of the presence (P-1150 vs P-1153) or absence (P-1148 vs P-1151) of ^{L}H34A substitution, the ^{H}F59Y CDR germlining substitution resulted in a consistent albeit modest reduction in both potency (EC₅₀; about 1.8-fold reduction) and signal (both Vmax and fold induction; 10-15% reduction). Compared to P-0734, the cumulative CDR germlining substitutions in P-1153 ended up with a nearly 20-fold decrease in PD1 blockade potency (EC₅₀) and a 50% reduction in Vmax. As a result, both ^{L}H34A and ^{H}F59Y substitutions were deemed detrimental in this particular framework and the original CDR residues, ^{L}His34 and ^{H}Phe59, will be preserved.

However, despite the notable differences in potency in blocking the PD1/PD-L1 interaction, all four antibodies and the Reference Antibody P-0734 displayed nearly identical binding strength with an EC₅₀ value close to 100 pM (FIG. 2A and Table 5).

TABLE 5
ELISA binding and PD1 blocking activity
of exemplary PD1 blocking antibodies

| | | PD1 blocking activity | | |
|---|---|---|---|---|
| | | (reporter assay) | | |
| | ELISA binding | EC₅₀ | Vmax | Fold |
| PD1 Ab | EC₅₀ (pM) | (nM) | (RLU) | induction |
| P-0734 | 82 | 0.53 | 58381 | 5.13 |
| P-1148 | 93 | 1.56 | 48227 | 4.63 |
| P-1150 | 88 | 5.55 | 36537 | 3.49 |
| P-1151 | 65 | 2.83 | 44252 | 4.15 |
| P-1153 | 110 | 9.88 | 30760 | 3.04 |

This piece of data suggested that the mechanistic-based functional assay is capable of discerning minute activity changes that aren't detectable by ELISA binding assay. As such, the luciferase PD1/PD-L1 reporter assay is herein used as the primary tool to characterize and rank PD1 blocking antibodies derived from pembrolizumab via germlining substitution. It is expected that the derivative antibodies that maintain full functional activity will exhibit identical in vivo efficacy as pembrolizumab.

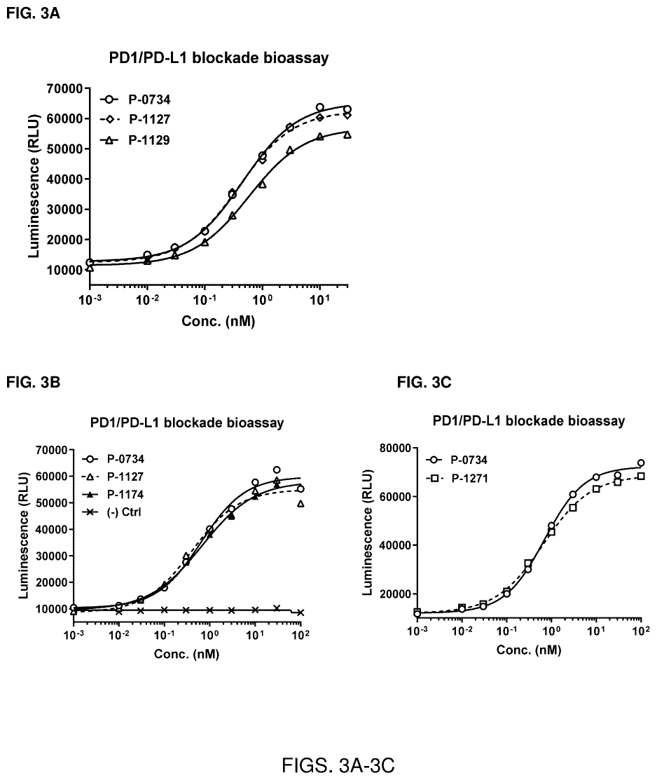

The potential negative effects of CDR-L2 germlining substitution, ^{L}E55A, was further assessed by comparing P-1127 and P-1129. Both these molecules contain ^{L}K27Q and ^{L}K54E germline substitutions in the light chain CDRs, with the only sequence difference being the extra CDR-L2 substitution, ^{L}E55A, in P-1129. As demonstrated in FIG. 3A, P-1129 displayed a minor yet appreciable reduction in potency (EC₅₀=0.39 nM and 0.58 nM for P-1127 and P-1129, respectively) and a marginal 10% decrease in maximal signal. Hence, the ^{L}E55A amino acid substitution was deemed adverse, and the original residues, ^{L}Glu55, will be preserved.

P-1174, harboring a total of 6 CDR germlining substitutions, ^{L}K27Q, ^{L}L54R, ^{H}N60A, ^{H}E61Q, ^{H}K64Q, and ^{H}N65G, exhibited identical PD1 blockade activity as P-1127 and the Reference Antibody P-0734 with EC₅₀ of 0.64 nM, 0.54 nM, and 0.67 nM for P-0734, P-1127, and P-1174, respectively (FIG. 3B). Additionally, P-1174 was derived from P-1148 by eliminating one CDR germlining substitution, ^{L}E55A. When compared to P-0734, P-1174 exhibited higher potency than P-1148 (refer to FIG. 2B & FIG. 3B). The data further collaborated the conclusion that the original CDR residues, ^{L}Glu55, should not be altered.

Besides the low conservation between the mouse CDRs and their human germline counterpart, multiple non-germline residues in the pembrolizumab VH framework also contributed to the low sequence similarity score with the germline. These non-germline residues, originating from the unique somatic mutations preserved in the acceptor framework sequence, including ^{H}Val9 in FR-1, ^{H}Thr76, ^{H}Lys82a, ^{H}Gln83, ^{H}Phe84 in FR-3 and ^{H}Thr108 in FR-4, are considered not to be structurally significant. To further enhance the sequence similarity score with the germline or degree of humanness, these f non-germline residues in the P-1174 framework were replaced with their respective germline equivalents, ^{H}V9A, ^{H}T76S, ^{H}K82aS, ^{H}Q83R, ^{H}F84S, ^{H}T108L, resulting in P-1271. As expected, P-1271 displayed the same PD1 blocking potency as the Reference Antibody P-0734 (FIG. 3C) with EC₅₀ values of 0.66 nM for P-1271 and 0.70 for P-0734, respectively.

In conclusion, CDR germlining substitutions, ^{L}K27Q, ^{L}L54R, ^{H}N60A, ^{H}E61Q, ^{H}K64Q, and ^{H}N65G, in P-1174 and P-1271 enhanced antibody sequence degree of humanness without compromising the potency in blocking the PD1/PD-L1 interaction. Additional six framework germlining substitutions in P-1271 further augmented the score of similarity to the closest human germline sequences. Table 6 lists the germlining substitutions and similarity scores to the closest human germline sequences of P-1174 and P-1271 in comparison to the Reference Antibody P-0734.

TABLE 6
Germlining substitutions and similarity scores of the exemplary optimized PD1
antibodies, P-1174 and P-1271, with the closest human germline sequences

| | | | VL similarity | VH similarity |
|---|---|---|---|---|
| | CDR germling | FR germlining | score to | score to |
| PD1 Ab | substitutions | substitutions | IGKV3D-11 | IGHV1-2 |
| P-0734 | N/A | N/A | 85.1% | 79.6% |
| P-1174 | ^{L}K27Q, ^{L}L54R, ^{H}N60A, | N/A | 87.2% | 83.7% |
| | ^{H}E61Q, ^{H}K64Q, and ^{H}N65G | | | |
| P-1271 | ^{L}K27Q, ^{L}L54R, ^{H}N60A, | ^{H}V9A, ^{H}T76S, | 87.2% | 88.8% |
| | ^{H}E61Q, ^{H}K64Q, and ^{H}N65G | ^{H}K82aS, ^{H}Q83R, | | |
| | | ^{H}F84S, ^{H}T108L | | |

### Example 5

### Evaluation of the PD1 Antibodies Comprising Germlining Modifications Based on the More Prevalent Human Germline Family (VH3)

The adoption of framework germlining substitutions based on the human antibody heavy chain germline IGHV3-23 (SEQ ID NO: 38) was investigated to exam whether an antibody framework with substantially lower sequence homology, but superior biophysical properties, could enhance the drug-like properties of the resultant antibody while fully retaining its functional activity. Of the 33 framework germlining substitutions (Table 7A), the importance of the 5 Vernier zone residues, ^{H}V48, ^{H}S49, ^{H}I69, ^{H}R71, and ^{H}N73, were assessed experimentally by reversion mutation to their respective pembrolizumab equivalents, ^{H}V48M, ^{H}S49G, ^{H}169L, ^{H}R71T, and ^{H}N73S, either individually or in combination. Additionally, six CDR-H2 residues, ^{H}F59Y, ^{H}N60A, ^{H}E61D, ^{H}K62S, ^{H}F63V, and ^{H}N65G, were selected for CDR germlining substitutions. Table 7B provides a summary of the VH3 germling substitutions in the exemplary antibodies.

TABLE 7A
VH Framework germling substitutions based on IGHV3-23

| VH FRs | Designation | Sequences |
|---|---|---|
| FR1 (1-30) | P-0734 | QVQLVQSGVEVKKPGASVKVSCKASGYTFT |
| | After Germlining | EVQLLESGGGLVQPGGSLRLSCAASGFTFT |
| FR2 (36-49) | P-0734 | WVRQAPGQGLEWMG |
| | After Germlining | WVRQAPGKGLEWVS |
| FR3 (66-94) | P-0734 | RVTLTTDSSTTTAYMELKSLQFDDTAVYYCAR |
| | After Germlining | RFTISRDNSKNTLYLQMNSLRAEDTAVYYCAR |
| FR4 (103-113) | P-0734 | WGQGTTVTVSS |
| | After Germlining | WGQGTLVTVSS |

The bolded and underlined residues represent a total of 33 framework germlining substitutions.

TABLE 7B
VH CDR germlining and FR reversion mutations based on IGHV3-23

| | CHR-H2 | No. of FR | Reversion |
|---|---|---|---|
| | germlining | germlining | mutation of |
| PD1 Ab | substitutions | substitutions | FR residues |
| P-1175 | ^{H}F59Y, ^{H}N60A, ^{H}E61D, ^{H}N65G. | 33 | N/A |
| P-1181 | ^{H}E61D, ^{H}K62S, ^{H}F63V, ^{H}N65G. | 33 | N/A |
| P-1176/ | ^{H}F59Y, ^{H}N60A, ^{H}E61D, ^{H}N65G | 30 | ^{H}I69L, ^{H}R71T, ^{H}N73S |
| P-1194 | | | |
| P-1177 | ^{H}F59Y, ^{H}N60A, ^{H}E61D, ^{H}N65G | 31 | ^{H}V48M, ^{H}S49G |
| P-1178/ | ^{H}F59Y, ^{H}N60A, ^{H}E61D, ^{H}N65G | 28 | ^{H}V48M, ^{H}S49G, |
| P-1238 | | | ^{H}I69L, ^{H}R71T, ^{H}N73S |
| P-1193 | ^{H}N60A, ^{H}E61D, ^{H}N65G | 33 | N/A |
| P-1198 | ^{H}N60A, ^{H}E61D, ^{H}N65G | 32 | ^{H}N73S |
| P-1199 | ^{H}N60A, ^{H}E61D, ^{H}N65G | 31 | ^{H}R71T, ^{H}N73S |
| P-1201 | ^{H}N60A, ^{H}E61D, ^{H}N65G | 30 | ^{H}I69L, ^{H}R71T, ^{H}N73S |

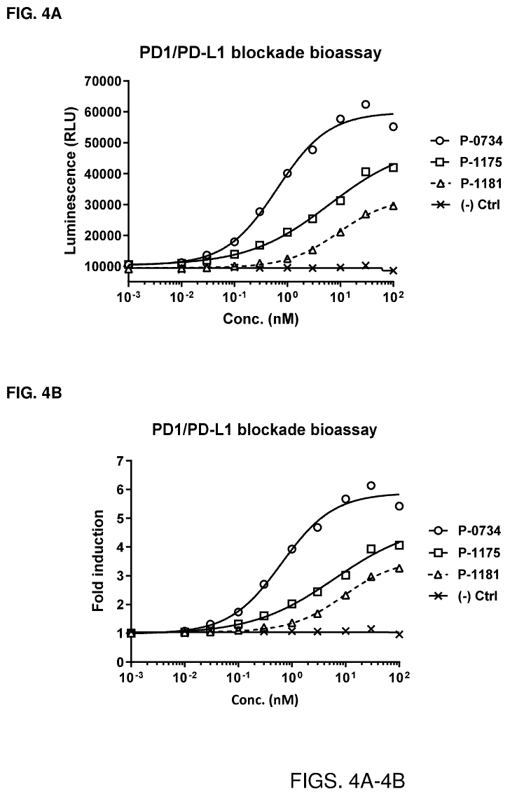

FIG. 4 depicts the PD1 blocking activity of P-1175 and P-1181, differing only in their CDR-H2 germlining substitutions (as shown in Table 7). Compared to the Reference Antibody P-0734, both P-1175 and P-1181 exhibited substantially diminished potency in blocking PD1 interaction. Specifically, P-1175 showed a 10-fold reduction in potency (EC₅₀) and 25% decrease in both Vmax and fold induction. This was in comparison to P-1181's 15-fold drop in potency and 40-50% decrease in Vmax and fold induction (as illustrated in FIGS. 4A & 4B and summarized in Table 8). Since P-1181 displayed a more drastic decline in activity, its two distinct CDR germlining substitutions, ^{H}K62S, ^{H}F63V, were deemed detrimental, hence the original CDR residues, ^{H}Lys62 and ^{H}Phe63, will be preserved. The findings suggested that the significance of individual CDR residues need to be assessed experimentally; even CDR residues that are close to the boundary or are not immediately adjacent to antigen-contacting residues could negatively impact the activity.

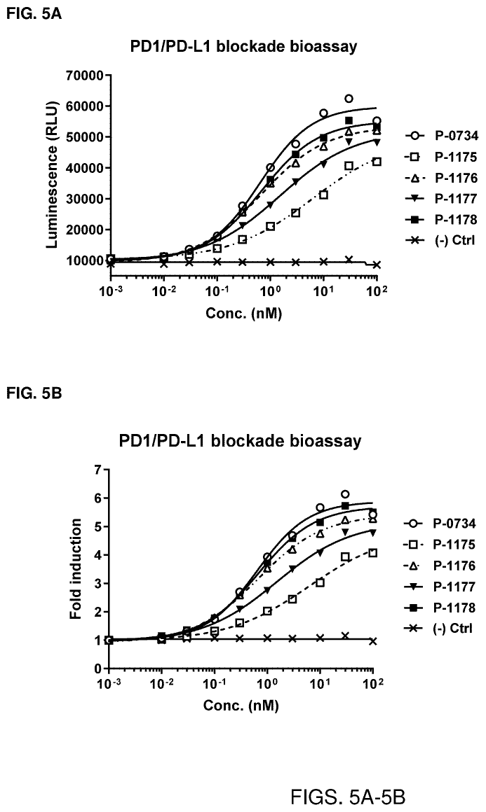

Two to five framework residue reversion mutations were introduced to P-1175, resulting in P-1176, P-1177, and P-1178, as detailed in Table 7. As indicated by the data in FIG. 5, the combined reversion mutations, ^{H}I69L, ^{H}R71T, and ^{H}N73S, in P-1176 essentially restored PD1 blocking activity to its original level. Similarly, the activity was significantly reinstated in P-1177 due to the combined reversion mutations, ^{H}V48M, and ^{H}S49G, although not as effectively as in P-1176. Nevertheless, the incorporation of these two reversion mutations (^{H}V48M and ^{H}S49G) into P-1176 did not lead to further enhancement in activity for the resulting antibody, P-1178 (P-1178 vs P-1176 in FIG. 5 and Table 8).

TABLE 8
PD1 blockade activity of exemplary PD1 blocking antibodies

| | PD1 blocking activity | | |
|---|---|---|---|
| | (reporter assay) | | |
| | EC₅₀ | Vmax | Fold |
| PD1 Ab | (nM) | (RLU) | induction |
| P-0734 | 0.64 | 55200 | 5.42 |
| P-1175 | 6.21 | 41970 | 4.07 |
| P-1181 | 9.18 | 29595 | 3.26 |
| P-1176 | 0.67 | 52093 | 5.27 |
| P-1177 | 1.51 | 48120 | 4.77 |
| P-1178 | 0.68 | 53089 | 5.50 |

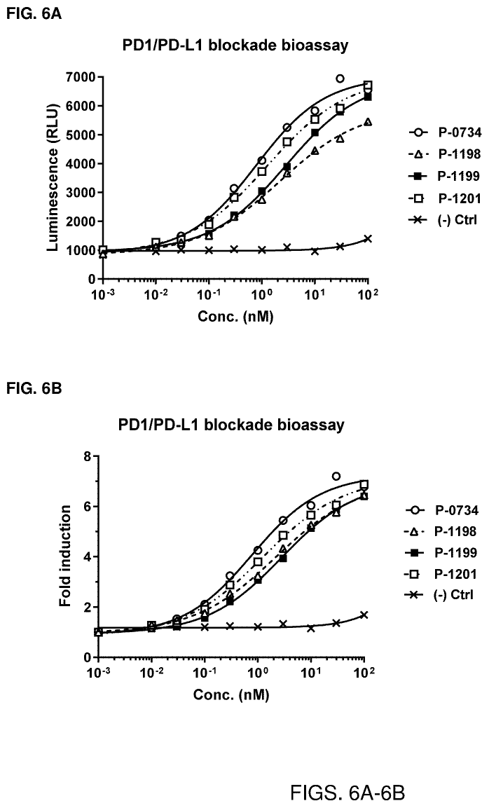

The significance of each of the three FR reversion mutations, ^{H}I69L, ^{H}R71T, and ^{H}N73S, were further assessed by comparing PD1 blocking activity of P-1198 (^{H}N73S), P-1199 (^{H}R71T, ^{H}N73S), and P-1201 (^{H}I69L, ^{H}R71T, ^{H}N73S). As demonstrated in FIG. 6, each added reversion mutation led to slight yet evident cumulative increases in PD1 inhibitory activity. Only the combination of all the three reversion mutations in P-1201 led to nearly fully restored functional activity (with EC₅₀ values of 1.28 nM and 0.78 nM for P-1201 and P-0734, respectively). Thus, all the three reversion mutations, ^{H}I69L, ^{H}R71T, ^{H}N73S, were deemed essential and will be incorporated.

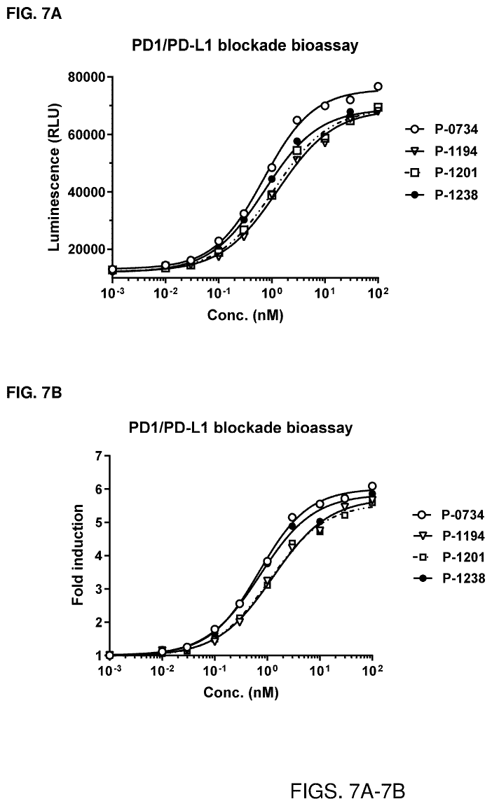

Further, the PD1 inhibitory activity of P-1194, P-1201, and P-1238 were compared and illustrated in FIGS. 7A and 7B. P-1194 and P-1201, differing by only one additional CDR germlining substitution, ^{H}F59Y, displayed identical PD1 blocking potency. This suggests that this particular substitution did not negatively affect the activity, contradicting earlier observation that ^{H}F59Y germlining substitution was detrimental when IGHV1-2 germline sequence was adopted. It is thus postulated that the impact of individual CDR germlining substitution is dependent on the context of the surrounding framework sequence. P-1238 were equally potent as the Reference Antibody with EC₅₀ values of 0.73 nM and 0.70 nM, respectively. Compared to P-1194, the two additional framework reversion mutations, ^{H}V48M, and ^{H}S49G, in P-1238 contributed to a slight but discernable improvement in activity.

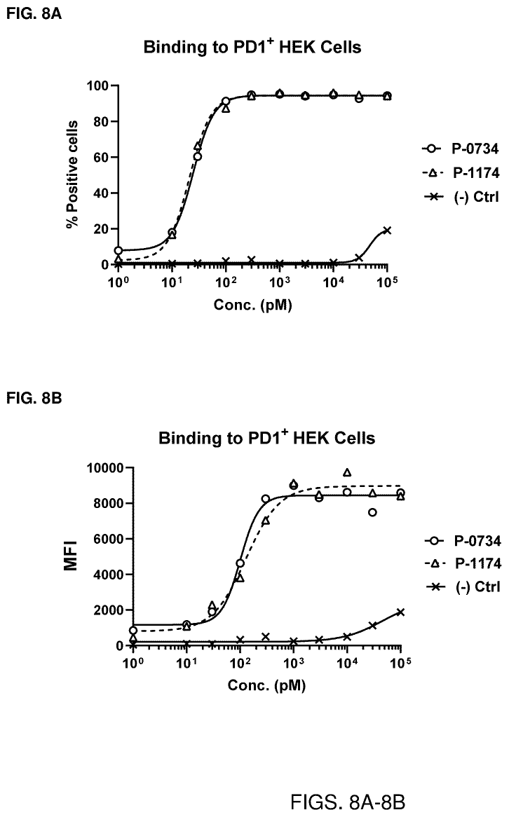

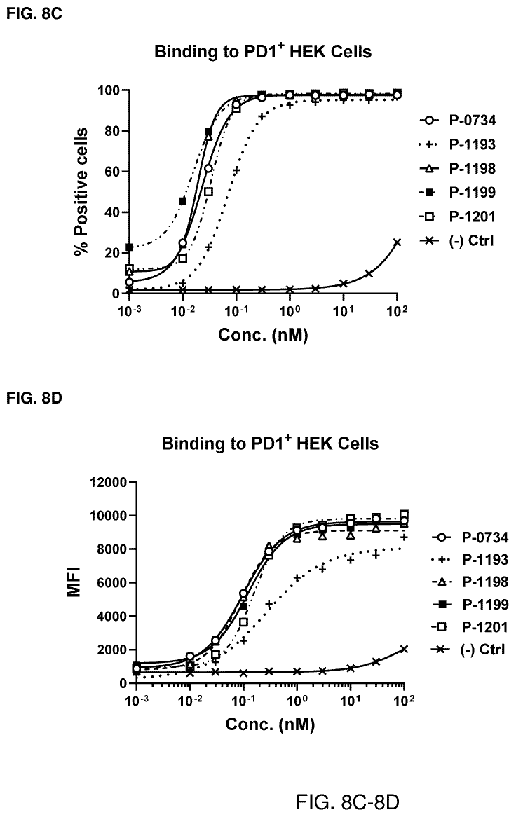

In the final assessment, P-1174, P-1193, P-1198, P-1199, and P-1201 were tested for their binding strength to PD1⁺ cells (FIG. 8). As anticipated, P-1174, which fully preserved PD1 blocking potency (FIG. 3C), displayed a binding affinity to PD1-expressing cells equivalent to the Reference Antibody, P-0734 (FIGS. 8A and 8B). P-1198, P-1199, and P-1201, which contain 1-3 framework reversion mutations, displayed subtle but evident potency difference in blocking PD1/PD-L1 interaction (FIG. 6), but such variations in activity were not detected in the cell-based binding assay. All three compounds demonstrated an equal ability in binding to PD1⁺ cells as P-0734 (FIGS. 8C & 8D and Table 9). Yest, the cell-based binding assay was able to distinguish P-1193, which contains no framework reversion mutation, from other compounds, as illustrated in FIGS. 8C & 8D and Table 9. The extent of the decrease, though, was less pronounced than what was observed in the blocking assay. The data further corroborate our earlier observation that the mechanistic-based PD1/PD-L1 blocking assay is more sensitive than binding assays in identifying subtle activity differences.

TABLE 9
Binding strength of exemplary optimized
PD1 blocking antibodies to PD1+ cells

PD1+ Cell binding
EC₅₀ (pM)

| PD1 Ab | % Positive cells | MFI |
|---|---|---|
| P-0734 | 23 | 97 |
| P-1193 | 70 | 259 |
| P-1198 | 19 | 87 |
| P-1199 | 16 | 120 |
| P-1201 | 33 | 159 |

In summary, the optimized PD1 blocking antibodies, P-1194, P-1201, and P-1238, built on a VH framework (IGHV3-23) that is of substantially lower sequence homology but superior biophysical properties, are able to fully or nearly fully retain antibody's functional activity and display improved similarity scores to the closest human germline sequence (IGHV3-23). The mutation details and similarity scores for each antibody are summarized in Table 10.

TABLE 10
CDR germlining substitutions, FR reversion mutations, and similarity scores
to the closest human germline sequences of exemplary optimized PD1 antibodies

| | | No. of FR | VH FR | VL Similarity | VH Similarity |
|---|---|---|---|---|---|
| | CDR germling | germlining | reversion | Score to | Score to |
| PD1 Ab | substitutions | substitutions | mutations | IGKV3D-11 | IGHV3-23 |
| P-0734 | N/A | N/A | N/A | 85.1% | 51.0% |
| P-1194 | ^{L}K27Q, ^{L}L54R, | 30 | ^{H}I69L, ^{H}R71T, | 87.2% | 83.7% |
| | ^{H}F59Y, ^{H}N60A, | | ^{H}N73S | | |
| | ^{H}E61D, ^{H}N65G | | | | |
| P-1201 | ^{L}K27Q, ^{L}L54R, | 30 | ^{H}I69L, ^{H}R71T, | 87.2% | 82.7% |
| | ^{H}N60A, ^{H}E61D, | | ^{H}N73S | | |
| | ^{H}N65G | | | | |
| P-1238 | ^{L}K27Q, ^{L}L54R, | 28 | ^{H}V48M, | 87.2% | 81.6% |
| | ^{H}F59Y, ^{H}N60A, | | ^{H}S49G, ^{H}I69L, | | |
| | ^{H}E61D, ^{H}N65G | | ^{H}R71T, ^{H}N73S | | |

### Example 6

### Germlining Substitutions Let to Decreased Hydrophobicity of the Optimized PD1 Blocking Antibodies

Among the 23 FDA and EMA approved therapeutic mAbs, pembrolizumab was the most hydrophobic one and consequently had the highest tendency to aggregate (Goyon et al., J. Chromatogr. B 1065-1066:35-43, 2017). Consistent with the experimentally determined apparent hydrophobic interaction chromatography (HIC) retention factors (k), the SSH2.0 hydrophobic prediction tool (http://i.uestc.edu.cn/SSH2/; Zhou et al., Front. Genet. 13:842127, 2022) indicated that both variable chains of pembrolizumab carry a significant risk of hydrophobic interaction. The probabilities of hydrophobic interaction risk for VH and VL are 0.97 and 0.61, respectively. An antibody is predicted to have a high risk of hydrophobic interaction if the probability is 0.5 or more (with 1 being the maximum possible value).

While the focus of the germlining substitutions was to enhance the degree of ‘humanness” in the antibody sequence, the process also resulted in a significant reduction in the probabilities of hydrophobic interaction for multiple optimized antibody sequences. Table 11 provides a summary of the predicted probabilities of hydrophobic interaction for the variable domains of the exemplary optimized PD1 blocking antibody, as estimated by SSH2.0.

TABLE 11
Summary of exemplary antibodies with improved similarity
scores and reduced probabilities for hydrophobicity
interaction while retaining PD1 blocking activity

| | SEQ ID NOS | | Similarity score to | | Probability of | | PD1 |
|---|---|---|---|---|---|---|---|
| | Light | Heavy | human germline | | hydrophobic interaction | | blocking |
| PD1 Ab | chain | chain | VL | VH | VL | VH | activity |
| P-0734 | 42 | 43 | 85.1% | 79.6% | 0.607 | 0.971 | Reference |
| P-1174 | 44 | 45 | 87.2% | 83.7% | 0.131 | 0.848 | Fully retain |
| P-1271 | 44 | 49 | 87.2% | 88.8% | 0.131 | 0.185 | Fully retain |
| P-1194 | 44 | 46 | 87.2% | 83.7% | 0.131 | 0.794 | Almost fully |
| | | | | | | | retain |
| P-1201 | 44 | 47 | 87.2% | 82.7% | 0.131 | 0.794 | Almost fully |
| | | | | | | | retain |
| P-1238 | 44 | 48 | 87.2% | 81.6% | 0.131 | 0.740 | Fully retain |

As shown in Table 11, the two light chain CDR germlining substitutions, ^{L}K27Q and ^{L}L54R, markedly decreased the hydrophobicity probability of VL from 0.607 (for Reference Antibody P-0734) to 0.131. These two amino acid changes were applied to the VL in all the optimized PD1 blocking antibodies listed in Table 11. The CDR germline substitutions in the heavy chain only resulted in a marginal reduction in hydrophobicity, with hydrophobicity probabilities altering from 0.971 (for Reference Antibody P-0734) to 0.848 (for P-1174) and to approximately 0.8 (for antibodies with HC based on VH-3 family frameworks, including P-1194, 1201, and 1238). However, when germlining substitutions (^{H}V9A, ^{H}T76S, ^{H}K82aS, ^{H}Q83R, ^{H}F84S, ^{H}T108L) were implemented in the HC framework of P-1174, the resulting construct, P-1271, had a significantly reduced hydrophobicity probability of 0.185, much lower than that of P-1174.

Hydrophobic patches on an antibody's surface are often implicated as one of the main contributions to its propensity to aggregate. Furthermore, these hydrophobic patches can cause high viscosity. consequently, the exemplary PD1 blocking antibodies, which have significantly diminished hydrophobic potentials, are expected to exhibit improved biophysical properties. Bifunctional molecules, including bispecific antibodies and immunocytokines, constructed using these optimized PD1 blocking antibodies are also projected to have enhanced developability profiles.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### Sequence Listings

The amino acid sequences listed in the accompanying sequence listing are shown using one letter code for amino acids, as defined in 37 C.F.R. 1.822.

SEQ ID NO: 1 is the amino acid sequence of a mature human PD1 polypeptide.

SEQ ID NOS: 2-5 are the amino acid sequences of light chain variable domain of PD1 blocking antibodies.

SEQ ID NOS: 6-18 are the amino acid sequences of heavy chain variable domain of PD1 blocking antibodies.

SEQ ID NOS: 19-21 are the amino acid sequences of light chain CDR1 of PD1 blocking antibodies.

SEQ ID NOS: 22-24 are the amino acid sequences of light chain CDR2 of PD1 blocking antibodies.

SEQ ID NO: 25 is the amino acid sequence of light chain CDR3 of PD1 blocking antibody.

SEQ ID NO: 26 is the amino acid sequence of heavy chain CDR1 of PD1 blocking antibody.

SEQ ID NOS: 27-32 are the amino acid sequences of heavy chain CDR2 of PD1 blocking antibodies.

SEQ ID NO: 33 is the amino acid sequence of heavy chain CDR3 of PD1 blocking antibody.

SEQ ID NO: 34 is the amino acid sequence of human kappa light chain constant domain.

SEQ ID NO: 35 is the amino acid sequence of human IgG1 heavy chain constant domain comprising L234A/L235A/G237A mutations.

SEQ ID NO: 36 is the amino acid sequence of human IgG4 heavy chain constant domain comprising S228P mutation.

SEQ ID NO: 37 is the amino acid sequence of human immunoglobulin germline heavy chain exon HGHV1-2 (GenBank accession NO: X62106).

SEQ ID NO: 38 is the amino acid sequence of human immunoglobulin germline heavy chain exon HGHV3-23 (GenBank accession NO: M99660).

SEQ ID NO: 39 is the amino acid sequence of human immunoglobulin germline light chain exon HGKV3D-11 (GenBank accession NO: X17264).

SEQ ID NO: 40 is the amino acid sequence of human antibody heavy chain variable domain with GenBank accession NO: AB063829.

SEQ ID NO: 41 is the amino acid sequence of human antibody light chain variable domain with GenBank accession NO: M29469.

SEQ ID NO: 42 is the amino acid sequence of the light chain of Reference Antibody P-0734.

SEQ ID NO: 43 is the amino acid sequence of the heavy chain of Reference Antibody P-0734.

SEQ ID NO: 44 is the amino acid sequence of the light chain of PD1 blocking antibodies.

SEQ ID NO: 45 is the amino acid sequence of the heavy chain of PD1 blocking antibody P-1174.

SEQ ID NO: 46 is the amino acid sequence of the heavy chain of PD1 blocking antibody P-1194.

SEQ ID NO: 47 is the amino acid sequence of the heavy chain of PD1 blocking antibody P-1201.

SEQ ID NO: 48 is the amino acid sequence of the heavy chain of PD1 blocking antibody P-1238.

SEQ ID NO: 49 is the amino acid sequence of the heavy chain of PD1 blocking antibody P-1271.

1-27. (canceled)

28. An isolated PD1 blocking antibody, or antigen-binding fragment thereof, which specifically binds human PD-1, selected from the group consisting of an PD1 blocking antibody, or antigen-binding fragment thereof comprising (a) the light chain CDR1, CDR2, and CDR3 sequences set forth in SEQ ID NO: 20, SEQ ID NO: 23, and SEQ ID NO: 25 and the heavy chain CDR1, CDR2, and CDR3 sequences set forth in SEQ ID NO: 26, SEQ ID NO: 28, and SEQ ID NO: 33; or comprising (b) the light chain CDR1, CDR2, and CDR3 sequences set forth in SEQ ID NO: 20, SEQ ID NO: 23, and SEQ ID NO: 25 and the heavy chain CDR1, CDR2, and CDR3 sequences set forth in SEQ ID NO: 26, SEQ ID NO: 30, and SEQ ID NO: 33; or comprising (c) the light chain CDR1, CDR2, and CDR3 sequences set forth in SEQ ID NO: 20, SEQ ID NO: 23, and SEQ ID NO: 25 and the heavy chain CDR1, CDR2, and CDR3 sequences set forth in SEQ ID NO: 26, SEQ ID NO: 31, and SEQ ID NO: 33.

29. An isolated PD1 blocking antibody, or antigen-binding fragment thereof according to claim 28, further comprising a set of four variable region framework regions from a human immunoglobulin (IgG).

30. The isolated PD1 blocking antibody, or antigen-binding fragment thereof according to claim 28, wherein the antibody or antigen-binding fragment is selected from a humanized antibody, a monoclonal antibody, a recombinant antibody, a single chain antibody, a diabody, a triabody, a tetrabody, a Fab fragment, a Fab′ fragment, a Fab₂ fragment, a F (ab)′₂ fragment, a domain antibody, an IgD antibody, an IgE antibody, an IgM antibody, an IgG1 antibody, an IgG2 antibody, an IgG3 antibody, an IgG4 antibody, or an IgG1 antibody harboring mutations in the Fc region that abolish the Fc effector function.

31. An isolated PD1 blocking antibody, or antigen-binding fragment thereof according to claim 30, selected from the group consisting of an PD1 blocking antibody, or antigen-binding fragment thereof comprising (a) a light chain variable region comprising amino acids having the sequence set forth in SEQ ID NO: 3, and a heavy chain variable region comprising amino acids having the sequence set forth in SEQ ID NO: 7; or comprising (b) a light chain variable region comprising amino acids having the sequence set forth in SEQ ID NO: 3, and a heavy chain variable region comprising amino acids having the sequence set forth in SEQ ID NO: 9; or comprising (c) a light chain variable region comprising amino acids having the sequence set forth in SEQ ID NO: 3, and a heavy chain variable region comprising amino acids having the sequence set forth in SEQ ID NO: 11; or comprising (d) a light chain variable region comprising amino acids having the sequence set forth in SEQ ID NO: 3, and a heavy chain variable region comprising amino acids having the sequence set forth in SEQ ID NO: 13; or comprising (e) a light chain variable region comprising amino acids having the sequence set forth in SEQ ID NO: 3, and a heavy chain variable region comprising amino acids having the sequence set forth in SEQ ID NO: 18.

32. An isolated PD1 blocking antibody, or antigen-binding fragment thereof according to claim 31, wherein the PD1 blocking antibody, or antigen-binding fragment thereof comprises a light chain variable region comprising amino acids having the sequence set forth in SEQ ID NO: 3, and a heavy chain variable region comprising amino acids having the sequence set forth in SEQ ID NO: 7.

33. An isolated PD1 blocking antibody, or antigen-binding fragment thereof according to claim 31, wherein the PD1 blocking antibody, or antigen-binding fragment thereof comprises a light chain variable region comprising amino acids having the sequence set forth in SEQ ID NO: 3, and a heavy chain variable region comprising amino acids having the sequence set forth in SEQ ID NO: 9.

34. An isolated PD1 blocking antibody, or antigen-binding fragment thereof according to claim 31, wherein the PD1 blocking antibody, or antigen-binding fragment thereof comprises a light chain variable region comprising amino acids having the sequence set forth in SEQ ID NO: 3, and a heavy chain variable region comprising amino acids having the sequence set forth in SEQ ID NO: 13.

35. An isolated humanized PD1 blocking antibody, or antigen-binding fragment thereof according to claim 30, wherein the PD1 antibody, or antigen-binding fragment thereof comprises a light chain having a sequence set forth in SEQ ID NO: 44, and a heavy chain having the sequence selected from the group consisting of the sequences set forth in SEQ ID NOs: 45-49.

36. An isolated humanized PD1 blocking antibody or antigen-binding fragment thereof according to claim 32, selected from the group consisting of an PD1 blocking antibody, or antigen-binding fragment thereof comprising (a) the light chain sequence set forth in SEQ ID NO: 44, and the heavy chain sequence set forth in SEQ ID NO: 45; or comprising (b) the light chain sequence set forth in SEQ ID NO: 44, and the heavy chain sequence set forth in SEQ ID NO: 46; or comprising (c) the light chain sequence set forth in SEQ ID NO: 44, and the heavy chain sequence set forth in SEQ ID NO: 47; or comprising (d) the light chain sequence set forth in SEQ ID NO: 44, and the heavy chain sequence set forth in SEQ ID NO: 48; or comprising (e) the light chain sequence set forth in SEQ ID NO: 44, and the heavy chain sequence set forth in SEQ ID NO: 49.

37. An isolated humanized PD1 blocking antibody, or antigen-binding fragment thereof according to claim 36, wherein the humanized PD1 blocking antibody, or antigen-binding fragment thereof comprises a light chain sequence set forth in SEQ ID NO: 44, and a heavy chain sequence set forth in SEQ ID NO: 45.

38. An isolated humanized PD1 blocking antibody, or antigen-binding fragment thereof according to claim 36, wherein the humanized PD1 blocking antibody, or antigen-binding fragment thereof comprises a light chain sequence set forth in SEQ ID NO: 44, and a heavy chain sequence set forth in SEQ ID NO: 48.

39. An isolated humanized PD1 blocking antibody, or antigen-binding fragment thereof according to claim 36, wherein the humanized PD1 blocking antibody, or antigen-binding fragment thereof comprises a light chain sequence set forth in SEQ ID NO: 44, and a heavy chain sequence set forth in SEQ ID NO: 49.

40. A pharmaceutical composition comprising an isolated PD-1 blocking antibody or antigen-binding fragment thereof according to claim 28 in admixture with a pharmaceutically acceptable carrier.

41. A method for enhancing the immune response to cancerous cells in a subject, comprising administering to said subject a therapeutically effective amount of the pharmaceutical composition of claim 40.

42. A method of treating cancerous cells in a subject, comprising administering to said subject a therapeutically effective amount (either as monotherapy or in a combination therapy regimen) of the pharmaceutical composition of claim 40.

43. The method according to claim 42, wherein the cancerous cell is selected from the group consisting of ovarian cancer, lung cancer, breast cancer, gastric cancer, prostate cancer, colon cancer, renal cell cancer, glioblastoma, and melanoma, and wherein the cancerous cell is associated with elevated expression of PD1.

44. An isolated immunoconjugate or fusion protein comprising an antibody or antigen-binding fragment thereof according to claim 28 coupled to an effector molecule, wherein the effector molecule is an immunotoxin, cytokine, chemokine, therapeutic agent, or a chemotherapeutic agent.

45. An isolated nucleic acid comprising a polynucleotide sequence encoding an antibody or antigen-binding fragment thereof according to claim 28.

46. A recombinant expression vector comprising the isolated nucleic acid of claim 45.

47. A host cell comprising the vector of claim 46.
